(19) United States
(12) Patent Application Publication
(10) Pub. No.: US 2019/0345520 A1
(43) Pub. Date: Nov. 14, 2019

(54) METHODS OF PRODUCING PHYTOENE

(71) Applicant: DEINOVE, GRABELS (FR)

(72) Inventor: JEAN-PAUL LEONETTI, CASTELNAU LE LEZ (FR)

(21) Appl. No.: 16/470,280

(22) PCT Filed: Dec. 15, 2017
(86) PCT No.: PCT/EP2017/083116

§ 371 Date: Jun. 17, 2019

(30) Foreign Application Priority Data
Dec. 16, 2016 (EP) 16306716.8

(51) Int. Cl. C12P 5/00; C12N 9/02; C12N 9/10; C12N 9/90; C07C 11/02

(52) U.S. Cl. CPC C12P 5/007; C12Y 205/01032; C12Y 103/99031; C12N 9/001; C07C 11/02; C12Y 202/01007; C12Y 503/03002; C12N 9/1022; C12N 9/90; C12N 9/1085

(57) ABSTRACT

The present invention relates to a recombinant Deinococcus bacterium genetically modified to produce and accumulate phytoene, and its use for producing phytoene. In particular, the present invention relates to a method of producing phytoene using a genetically modified Deinococcus bacterium.

### FIELD OF THE INVENTION

The present invention relates to the field of microbiology and in particular to the field of biosynthetic pathway engineering. More specifically, the present invention relates to the field of production of phytoene using genetically modified bacteria.

### BACKGROUND OF THE INVENTION

Carotenoids are a class of natural pigments that are synthesized by all photosynthetic organisms and in some heterotrophic growing bacteria and fungi. Because animals are unable to synthetize de novo these molecules, carotenoids have been widely used commercially as food supplements, animal feed additives or nutraceuticals. They have also found various applications as colorants or for cosmetic and pharmaceutical purposes.

Although colored carotenoids are most extensively studied, colorless carotenoid such as phytoene and phytofluene have shown similar effective and benefiting activities. These carotenoids are found in majority of fruits and vegetables and may act as UV absorbers, antioxidants, and anti-inflammatory agents. As a consequence, they were found to be useful in cosmetics, nutrition and therapeutics, in particular in the treatment of skin disorders.

Phytoene (7,8,11,12,7′,8′,11′,12′-octahydro-ψ,ψ-carotene) is the first carotenoid in the carotenoid biosynthesis pathway and is produced by the dimerization of a 20-carbon atom precursor, geranylgeranyl pyrophosphate (GGPP). This reaction is catalyzed by the enzyme phytoene synthase. As precursor, phytoene is then desaturated to form successively phytofluene, ζ-carotene, neurosporene and finally lycopene.

Because they are precursors of all the others carotenoids, phytofluene and phytoene have been extensively studied in investigations dealing with the biosynthesis of these compounds. However, they have been largely neglected in other kinds of studies. As a consequence, to date, no current methods are available for producing phytoene via any biological process, and in particular for producing phytoene exempt of phytofluene.

### SUMMARY OF THE INVENTION

The present invention relates to a recombinant Deinococcus bacterium which is genetically modified to produce and accumulate substantial amount of phytoene, preferably exempt of phytofluene, and the use of said recombinant bacterium to produce phytoene.

Accordingly, in a first aspect, the present invention relates to a method of producing phytoene comprising culturing a recombinant Deinococcus bacterium under conditions suitable to produce phytoene, and optionally recovering said phytoene, wherein said recombinant Deinococcus bacterium is genetically modified to exhibit increased phytoene synthase activity and decreased phytoene desaturase activity.

Preferably, the recombinant Deinococcus bacterium does not exhibit any phytoene desaturase activity. The recombinant Deinococcus bacterium may be genetically modified by inactivating a gene encoding a phytoene desaturase, preferably by deleting all or part of said gene or introducing a nonsense codon, a cassette, a gene or a mutation inducing a frameshift. Preferably, the recombinant Deinococcus bacterium is genetically modified by deleting all or part of a gene encoding a phytoene desaturase.

The recombinant Deinococcus bacterium may also be genetically modified to overexpress a native gene encoding phytoene synthase, to express a heterologous gene encoding phytoene synthase, to express a native gene encoding phytoene synthase and comprising a mutation improving phytoene synthase activity of the encoded enzyme, or to express a gene encoding a feedback resistant phytoene synthase.

In some embodiments, the recombinant Deinococcus bacterium may further exhibit increased FPP synthase activity, increased DXP synthase and/or IPP isomerase activities, preferably increased FPP synthase, DXP synthase and IPP isomerase activities.

Preferably, the recombinant Deinococcus bacterium is a Deinococcus bacterium selected from the group consisting of D. geothermalis, D. murrayi, D. grandis, D. aquaticus, D. indicus, D. cellulosilyticus, D. depolymerans and D. maricopensis. More preferably, the recombinant Deinococcus bacterium is a Deinococcus geothermalis bacterium.

The recombinant Deinococcus bacterium is preferably able to produce at least 20 mg/g DCW (dry cell weight) of phytoene, in particular when cultured in aerobiosis and in the presence of glucose as carbon source.

Preferably, the recombinant Deinococcus bacterium produces only one isomer of phytoene which is 15-cis phytoene, and does not produce phytofluene, ζ-carotene, neurosporene or lycopene.

In some embodiments, the recombinant Deinococcus bacterium is a thermophilic Deinococcus, preferably D. geothermalis, and the culture of the recombinant Deinococcus bacterium under conditions suitable to produce phytoene is performed at a temperature comprised between 40 and 50° C., preferably between 45 and 48° C.

In a second aspect, the present invention also relates to the recombinant Deinococcus bacterium used in the method of the invention, or to a cell extract thereof, preferably a fraction comprising cell membranes. Preferably, said cell extract comprises phytoene and does not comprise any detectable amount of phytofluene, ζ-carotene, neurosporene or lycopene. Preferably the cell extract of the invention comprises only one isomer of phytoene which is 15-cis phytoene.

In another aspect, the present invention also relates to the use of the recombinant Deinococcus bacterium of the invention to produce phytoene.

In a further aspect, the present invention also relates to a composition comprising phytoene obtained by the method of the invention, preferably only 15-cis phytoene, wherein said composition does not comprise any detectable amount of phytofluene, ζ-carotene, neurosporene or lycopene. Preferably, said composition is a cosmetic, pharmaceutical or nutraceutical, nutricosmetic composition or a food or feed additive.

### DETAILED DESCRIPTION OF THE INVENTION

Deinococcus bacteria are non-pathogen bacteria that were firstly isolated in 1956 by Anderson and collaborators. These extremophile organisms have been proposed for use in industrial processes or reactions using biomass (see e.g., WO2009/063079; WO2010/094665 or WO2010/081899). Based on their solid knowledge of Deinococcus metabolism and genetics, the inventors found that Deinococcus bacteria can be genetically modified to produce substantial amounts of phytoene under conditions compatible with large scale production. Furthermore, they showed that this recombinant bacterium is able to produce phytoene exempt of phytofluene, ζ-carotene, neurosporene or lycopene thereby suppressing the need of further purification steps.

Definitions

In the context of the invention, the term “Deinococcus” includes wild type or natural variant strains of Deinococcus, e.g., strains obtained through accelerated evolution, mutagenesis, by DNA-shuffling technologies, or recombinant strains obtained by insertion of eukaryotic, prokaryotic and/or synthetic nucleic acid(s). Deinococcus bacteria can designate any bacterium of the genus Deinococcus, such as without limitation. D. actinosclerus, D. aerius, D. aerolatus, D. aerophilus, D. aetherius, D. alpinitundrae, D. altitudinis, D. antarcticus, D. apachensis, D. aquaticus, D. aquaticus, D. aquatilis, D. aquiradiocola, D. caeni, D. carri, D. cellulosilyticus, D. citri, D. claudionis, D. daejeonensis , D. depolymerans, D. desertii, D. enclensis, D. ficus, D. frigens, D. geothermalis, D. gobiensis, D. grandis, D. guangriensis, D. guilhemensis, D. hohokamensis, D. hopiensis, D. humi , D. indicus, D. maricopensis, D. marmoris, D. metalli, D. metallilatus, D. misasensis, D. murrayi, D. navajonensis, D. papagonensis, D. peraridilitoris, D. phoenicis, D. pimensis, D. piscis, D. proteolyticus, D. puniceus, D. radiodurans, D. radiomollis, D. radiophilus, D. radiopugnans, D. radioresistens, D. radiotolerans, D. reticulitermitis, D. roseus, D. sahariens, D. saxicola, D. soli, D. sonorensis, D. swuensis, D. wulumuqiensis, D. xinjiangensis, D. xibeiensis and D. yavapaiensis bacterium, or any combinations thereof. Preferably, the term “Deinococcus” refers to D. geothermalis, D. murrayi, D. grandis, D. aquaticus, D. indicus, D. cellulosilyticus, D. depolymerans or D. maricopensis. More preferably, the term “Deinococcus” refers to D. geothermalis, D. murrayi or D. maricopensis. Even more preferably, the term “Deinococcus” refers to D. geothermalis.

The terms “recombinant bacterium” and “genetically modified bacterium” or “engineered bacterium” are herein used interchangeably and designate a bacterium that 25 is not found in nature and which contains a modified genome as a result of either a deletion, insertion or modification of one or several genetic elements.

A “recombinant nucleic acid” designates a nucleic acid which has been engineered and is not found as such in wild type bacteria. In some particular embodiments, this term may refer to a gene operably linked to a promoter that is different from its naturally occurring promoter.

The term “gene” designates any nucleic acid encoding a protein. The term gene encompasses DNA, such as cDNA or gDNA, as well as RNA. The gene may be first prepared by e.g., recombinant, enzymatic and/or chemical techniques, and subsequently replicated in a host cell or an in vitro system. The gene typically comprises an open reading frame encoding a desired protein. The gene may contain additional sequences such as a transcription terminator or a signal peptide.

The term “operably linked” means a configuration in which a control sequence is placed at an appropriate position relative to a coding sequence, in such a way that the control sequence directs expression of the coding sequence.

The term “control sequences” means nucleic acid sequences necessary for expression of a gene. Control sequences may be native or heterologous. Well-known control sequences and currently used by the person skilled in the art will be preferred. Such control sequences include, but are not limited to, a leader, polyadenylation sequence, propeptide sequence, promoter, signal peptide sequence, and transcription terminator. Preferably, the control sequences include a promoter and a transcription terminator.

The term “expression cassette” denotes a nucleic acid construct comprising a coding region, i.e. a gene, and a regulatory region, i.e. comprising one or more control sequences, operably linked. Preferably, the control sequences are suitable for Deinococcus host cells.

As used herein, the term “expression vector” means a DNA or RNA molecule that comprises an expression cassette. Preferably, the expression vector is a linear or circular double stranded DNA molecule.

As used herein, the term “native” or “endogenous”, with respect to a bacterium, refers to a genetic element or a protein naturally present in said bacterium. The term “heterologous”, with respect to a bacterium, refers to a genetic element or a protein that is not naturally present in said bacterium.

The terms “overexpression” and “increased expression” as used herein, are used interchangeably and mean that the expression of a gene or an enzyme is increased compared to a non modified bacterium, e.g. the wild-type bacterium or the corresponding bacterium that has not been genetically modified in order to produce phytoene. Increased expression of an enzyme is usually obtained by increasing expression of the gene encoding said enzyme. In embodiments wherein the gene or the enzyme is not naturally present in the bacterium of the invention, i.e. heterologous gene or enzyme, the terms “overexpression” and “expression” may be used interchangeably. To increase the expression of a gene, the skilled person can used any known techniques such as increasing the copy number of the gene in the bacterium, using a promoter inducing a high level of expression of the gene, i.e. a strong promoter, using elements stabilizing the corresponding messenger RNA or modifying Ribosome Binding Site (RBS) sequences and sequences surrounding them. In particular, the overexpression may be obtained by increasing the copy number of the gene in the bacterium. One or several copies of the gene may be introduced into the genome by methods of recombination, known to the expert in the field, including gene replacement or multicopy insertion in IS sequences (see for example the international patent application WO 2015/092013). Preferably, an expression cassette comprising the gene, preferably placed under the control of a strong promoter, is integrated into the genome. Alternatively, the gene may be carried by an expression vector, preferably a plasmid, comprising an expression cassette with the gene of interest preferably placed under the control of a strong promoter. The expression vector may be present in the bacterium in one or several copies, depending on the nature of the origin of replication. The overexpression of the gene may also obtained by using a promoter inducing a high level of expression of the gene. For instance, the promoter of an endogenous gene may be replaced by a stronger promoter, i.e. a promoter inducing a higher level of expression. The promoters suitable to be used in the present invention are known by the skilled person and can be constitutive or inducible, and native or heterologous.

As used herein, the term “increased activity” refers to an enzymatic activity that is increased compared to a non modified bacterium, e.g. the wild-type bacterium or the corresponding bacterium that has not been genetically modified in order to produce or accumulate phytoene. This increase may be obtained for example by overexpression of a native gene or expression of a heterologous gene.

As used herein, the term “decreased activity” refers to an enzymatic activity that is decreased compared to a non modified bacterium, e.g. the wild-type bacterium or the corresponding bacterium that has not been genetically modified in order to produce or accumulate phytoene. This decrease may be obtained for example by inactivating the gene encoding the enzyme responsible of such activity.

As used herein, the term “sequence identity” or “identity” refers to the number (%) of matches (identical amino acid residues) in positions from an alignment of two polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithms (e.g. Needleman and Wunsch algorithm; Needleman and Wunsch, 1970) which aligns the sequences optimally over the entire length, while sequences of substantially different lengths are preferably aligned using a local alignment algorithm (e.g. Smith and Waterman algorithm (Smith and Waterman, 1981) or Altschul algorithm (Altschul et al., 1997; Altschul et al., 2005)). Alignment for purposes of determining percent amino acid sequence identity can be achieved in various ways that are within the skill in the art, for instance, using publicly available computer software available on internet web sites such as http://blast.ncbi.nlm.nih.gov/ or http://www.ebi.ac.uk/Tools/emboss/). Those skilled in the art can determine appropriate parameters for measuring alignment, including any algorithms needed to achieve maximal alignment over the full length of the sequences being compared. For purposes herein, % amino acid sequence identity values refers to values generated using the pair wise sequence alignment program EMBOSS Needle that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5

The terms “low stringency”, “medium stringency”, “medium/high stringency”, “high stringency” and “very high stringency” refer to conditions of hybridization. Suitable experimental conditions for determining hybridization between a nucleotide probe and a homologous DNA or RNA sequence involves presoaking of the filter containing the DNA fragments or RNA to hybridize in 5×SSC (Sodium chloride/Sodium citrate for 10 min, and prehybridization of the filter in a solution of 533 SSC, 5× Denhardt's solution, 0.5% SDS and 100 μg/ml of denatured sonicated salmon sperm DNA, followed by hybridization in the same solution containing a concentration of 10 ng/ml of a random-primed ³²P-dCTP-labeled (specific activity>1×10⁹ cpm/ng) probe for 12 hours at ca. 45° C. (Feinberg and Vogelstein, 1983). For various stringency conditions the filter is then washed twice for 30 minutes in 2×SSC, 0.5% SDS and at least 55° C. (low stringency), more preferably at least 60° C. (medium stringency), still more preferably at least 65° C. (medium/high stringency), even more preferably at least 70° C. (high stringency), and even more preferably at least 75° C. (very high stringency).

As used in this specification, the term “about” refers to a range of values ±10% of the specified value. For example, “about 20” includes ±10% of 20, or from 18 to 22. Preferably, the term “about” refers to a range of values ±5% of the specified value.

As used herein, the term “CrtB” or “phytoene synthase” refers to a phytoene synthase enzyme (EC 2.5.1.32) encoded by a crtB gene which catalyzes the condensation of two molecules of geranylgeranyl diphosphate (GGPP) to give phytoene.

The term “Crtl” or “phytoene desaturase” or “phytoene dehydrogenase” refers to a phytoene desaturase enzyme (EC 1.3.99.31) encoded by a crtl gene which catalyses up to four desaturation steps (EC 1.3.99.28 [phytoene desaturase (neurosporene-forming)], EC 1.3.99.29 [phytoene desaturase (zeta-carotene-forming)] and EC 1.3.99.30 [phytoene desaturase (3,4-didehydrolycopene-forming)]). In preferred embodiments, the phytoene desaturase enzyme converts phytoene to neurosporene and lycopene via the ntermediary of phytofluene and zeta-carotene.

According to the organism, the nomenclature of the above identified enzymes and encoding genes may vary. However, for the sake of clarity, in the present specification, these terms are used independently from the origin of the enzymes or genes.

As used herein, the term “phytoene” refers to any phytoene isomer, and preferably to 15-cis phytoene.

In a first aspect, the present invention relates to a recombinant Deinococcus bacterium which is genetically modified to exhibit increased phytoene synthase activity and decreased phytoene desaturase activity. The inventors indeed showed that such bacterium may produce and accumulate substantial amount of phytoene, exempt of phytofluene, without altering the bacterial viability or growth.

The recombinant Deinococcus bacterium of the invention is a Deinococcus bacterium which has been genetically modified in order to increase its production and accumulation of phytoene. By comparison to a wild-type bacterium, this recombinant bacterium may comprise genetic modifications as described above but also further modifications which are not directly linked to the production of phytoene but provide advantages for the industrial production of phytoene such as antibiotic resistance or enzymatic activities to enlarge the substrate range.

In the recombinant Deinococcus bacterium of the invention, the phytoene synthase activity is increased by comparison to the non modified bacterium.

The phytoene synthase activity may be determined by any method known by the skilled person. For example, said activity may be assessed by incubating phytoene synthase with GGPP, recovering the phytoene, and quantifying the phytoene by liquid scintillation counting (see e.g. Welsch et al., The Plant Cell, Vol. 22: 3348-3356, 2010).

Preferably, increased phytoene synthase activity is obtained by genetically modifying the Deinococcus bacterium to overexpress an endogenous CrtB gene, to express a heterologous CrtB gene, and/or to express an improved variant of the endogenous phytoene synthase.

In particular, the recombinant Deinococcus bacterium of the invention may comprise a heterologous nucleic acid encoding a polypeptide exhibiting phytoene synthase activity and/or may overexpress an endogenous nucleic acid encoding a polypeptide exhibiting phytoene synthase activity.

To increase the expression of a gene (i.e. to overexpress a gene), the skilled person can used any known techniques such as increasing the copy number of the gene in the bacterium, using a promoter inducing a high level of expression of the gene, i.e. a strong promoter, using elements stabilizing the corresponding messenger RNA or modifying Ribosome Binding Site (RBS) sequences and sequences surrounding them.

In a particular embodiment, the overexpression is obtained by increasing the copy number of the gene in the bacterium. One or several copies of the gene may be introduced into the genome by methods of recombination, known to the expert in the field, including gene replacement. Preferably, an expression cassette comprising the gene is integrated into the genome.

Alternatively, the gene may be carried by an expression vector, preferably a plasmid, comprising an expression cassette with the gene of interest. The expression vector may be present in the bacterium in 1 to 5, 20, 100 or 500 copies, depending on the nature of the origin of replication.

In another particular embodiment, the overexpression of the gene is obtained by using a promoter inducing a high level of expression of the gene. For instance, the promoter of an endogenous gene may be replaced by a stronger promoter, i.e. a promoter inducing a higher level of expression. The promoters suitable to be used in the present invention are known by the skilled person and can be constitutive or inducible, and native or heterologous.

Expression cassettes useful in the present invention comprising at least a CrtB gene operably linked to one or more control sequences, typically comprising a transcriptional promoter and a transcription terminator, that direct the expression of said gene.

A control sequence may include a promoter that is recognized by the host cell. The promoter contains transcriptional control sequences that mediate the expression of the enzyme. The promoter may be any polynucleotide that shows transcriptional activity in the Deinococcus bacterium. The promoter may be a native or heterologous promoter. Preferred promoters are native and Deinococcus promoters. In this regard, various promoters have been studied and used for gene expression in Deinococcus bacteria. Examples of suitable promoters include PtufA and PtufB promoters from the translation elongation factors Tu genes tufA (e.g., D. radiodurans: DR_0309) and tufB (e.g., D. radiodurans: DR_2050), the promoter of the resU gene located in pI3, the promoter region PgroESL of the groESL operon (Lecointe, et al. 2004. Mol Microbiol 53: 1721-1730; Meima et al. 2001. J Bacteriol 183: 3169-3175), or derivatives of such promoters. Preferably, the promoter is a strong constitutive promoter.

A control sequence may also comprise a transcription terminator, which is recognized by Deinococcus bacteria to terminate transcription. The terminator is operably linked to the 3′-terminus of the gene. Any terminator that is functional in Deinococcus bacteria may be used in the present invention such as, for example, the terminator term116 described in Lecointe, et al. 2004. Mol Microbiol 53: 1721-1730.

Optionally, the expression cassette may also comprise a selectable marker that permits easy selection of recombinant bacteria. Typically, the selectable marker is a gene encoding antibiotic resistance or conferring autotrophy.

In a particular embodiment, the recombinant Deinococcus bacterium of the invention comprises an expression cassette comprising a CrtB gene operably linked to a strong constitutive promoter.

The expression cassette may be integrated into the genome of the bacterium and/or may be maintained in an episomal form into an expression vector.

Preferably, the expression cassette is integrated into the genome of the bacterium. One or several copies of the expression cassette may be introduced into the genome by methods of recombination, known to the expert in the field, including gene replacement. The expression cassette can replace the endogenous CrtB gene or may be integrated in another place into the genome.

The expression cassette may also be integrated into the genome in order to inactivate target genes. In a particular embodiment, the expression cassette is integrated into the genome in order to inactivate a Crtl gene. In another embodiment, the expression cassette is integrated into the genome in order to inactivate the phosphotransacetylase (pta) gene. Targeted genes may be replaced or inactivated by the insertion of the cassette.

Alternatively, or in addition, the expression cassette may be integrated into the genome in a non-coding sequence, e.g. an insertion sequence (IS) (see for example the international patent application WO 2015/092013).

In embodiments wherein the expression cassette is maintained in an episomal form, the expression vector may be present in the bacterium in one or several copies, depending on the nature of the origin of replication.

The Deinococcus host cell may be transformed, transfected or transduced in a transient or stable manner. The recombinant Deinococcus bacterium of the invention may be obtained by any method known by the skilled person, such as electroporation, conjugation, transduction, competent cell transformation, protoplast transformation, protoplast fusion, biolistic “gene gun” transformation, PEG-mediated transformation, lipid-assisted transformation or transfection, chemically mediated transfection, lithium acetate-mediated transformation or liposome-mediated transformation.

The term “recombinant Deinococcus bacterium” also encompasses the genetically modified host cell as well as any progeny that is not identical to the parent host cell, in particular due to mutations that occur during replication.

The CrtB gene expressed or overexpressed in the recombinant bacterium of the invention may encode an endogenous phytoene synthase, a heterologous phytoene synthase or an improved variant of the endogenous phytoene synthase.

In particular, the polypeptide exhibiting phytoene synthase activity may be any known phytoene synthase, such as selected from known bacterial, algal or plant phytoene synthases.

The polypeptide exhibiting phytoene synthase activity may be selected from non Deinococcus phytoene synthases such as CrtB from Pantoea agglomerans (GenBank accession number: AFZ89043.1, SEQ ID NO: 1) or CrtB from Paracoccus sp_N81106 (GenBank accession number: BAE47469.1; SEQ ID NO: 2).

Preferably, the polypeptide exhibiting phytoene synthase activity may be selected from any phytoene synthase from Deinococcus bacteria.

Examples of Deinococcus phytoene synthases (CrtB) include, but are not limited to, phytoene synthases from D. geothermalis (Uniprot accession number: Q1J109; SEQ ID NO: 3), D. actinosclerus (Uniprot accession number: A0A0U3KC93; SEQ ID NO: 4), D. deserti (Uniprot accession number: C1D2Z3; SEQ ID NO: 5), D. gobiensis (Uniprot accession number: H8GYF6; SEQ ID NO: 6), D. maricopensis (Uniprot accession number: E8UAM8; SEQ ID NO: 7), D. peraridilitoris (Uniprot accession number: L0A567; SEQ ID NO: 8), D. puniceus (Uniprot accession number: A0A172TDE8; SEQ ID NO: 9), D. radiodurans (Uniprot accession number: Q9RW07; SEQ ID NO: 10), D. soli (Uniprot accession number: A0A0F7JV05; SEQ ID NO: 11) and D. swuensis (Uniprot accession number: A0A0A7KGT0; SEQ ID NO: 12).

The polypeptide exhibiting phytoene synthase activity may also be any polypeptide exhibiting phytoene synthase activity and having at least 60%, preferably 65, 70, 75, 80, 85, 90, 95, 96, 97, 98 or 99%, identity to any phytoene synthase listed above.

In a particular embodiment, the polypeptide exhibiting phytoene synthase activity is selected from the group consisting of

a) a polypeptide comprising, or consisting of, an amino acid sequence selected from the group consisting of SEQ ID NO: 1 to 12, preferably from the group consisting of SEQ ID NO: 3 to 12, more preferably SEQ ID NO: 3; and

b) a polypeptide exhibiting phytoene synthase activity and having an amino acid sequence having at least 60%, preferably 65, 70, 75, 80, 85, 90, 95, 96, 97, 98 or 99%, identity to any sequence selected from the group consisting of SEQ ID NO: 1 to 12, preferably to any sequence selected from the group consisting of SEQ ID NO: 3 to 12, more preferably to SEQ ID NO: 3.

The CrtB gene used in the present invention may also encode an improved phytoene synthase, i.e. an enzyme that possesses at least one mutation in its sequence, in comparison with the amino acid sequence of the wild-type enzyme, said mutation leading to an increase of its activity, an increased specific catalytic activity, an increased specificity for the substrate, an increased protein or RNA stability and/or an increased intracellular concentration of the enzyme, or leading to a feedback resistant mutant.

In an embodiment, the recombinant Deinococcus bacterium of the invention expresses a CrtB gene encoding an improved phytoene synthase, and in particular an improved Deinococcus phytoene synthase. Preferably, the improved Deinococcus phytoene synthase exhibits an increased catalytic activity.

In addition, in the recombinant Deinococcus bacterium of the invention, the endogenous phytoene desaturase activity is reduced by comparison to the non modified bacterium, preferably this activity is suppressed.

The phytoene desaturase activity may be determined by any method known by the skilled person. For example, said activity may be assessed by incubating phytoene desaturase with phytoene in the presence of catalase and glucose oxidase, adding a mixture of methanol and KOH and heating at 60° C. for 15 min to terminate the reaction, extracting the products from the incubation mixture with diethyl ether/light petroleum, evaporating the solvent phase and redissolving the residue in cool acetone/methanol, and identifying products by HPLC (see e.g. Xu et al., Microbiology, 153, 1642-1652, 2007).

In preferred embodiments, the recombinant Deinococcus bacterium of the invention does not produce significant or detectable amounts of phytofluene or any other intermediate molecules from phytoene to lycopene.

Preferably, the recombinant Deinococcus bacterium of the invention is genetically modified to inactivate a gene encoding a phytoene desaturase, a Crtl gene. This inactivation prevents conversion of phytoene to lycopene and thus leads to phytoene accumulation.

The Crtl gene may be inactivated by any method known by the skilled person, for example by deletion of all or part of this gene, by introducing a nonsense codon or a mutation inducing a frameshift, or by insertion of a gene or an expression cassette, e.g. a CrtB gene or an expression cassette comprising a CrtB genes.

Alternatively, the expression of the endogenous Crtl gene may be reduced. This reduction may be obtained, for example, by replacing endogenous promoters by weaker promoters, such as PlexA or PamyE promoters (Meima et al. 2001. J Bacteriol 183: 3169-3175).

In preferred embodiments, the Crtl gene is inactivated, preferably by deleting all or part of said gene, for example by gene replacement.

Examples of phytoene desaturases include, but are not limited to, phytoene desaturases of D. geothermalis (Uniprot accession number: Q1J108; SEQ ID NO: 13), D. actinosclerus (Uniprot accession number: A0A0U4CEJ5; SEQ ID NO: 14), D. deserti (Uniprot accession number: C1D2Z4; SEQ ID NO: 15), D. gobiensis (Uniprot accession number: H8GYF5; SEQ ID NO: 16), D. maricopensis (Uniprot accession number: E8UAM7; SEQ ID NO: 17), D. peraridilitoris (Uniprot accession number: LOA6E5; SEQ ID NO: 18), D. proteolyticus (Uniprot accession number: FORJ97; SEQ ID NO: 19), D. puniceus (Uniprot accession number: A0A172TAT8; SEQ ID NO: 20), D. radiodurans (Uniprot accession number: Q9RW08; SEQ ID NO: 21), D. soli (Uniprot accession number: A0AOF7JTT9; SEQ ID NO: 22) and D. swuensis (Uniprot accession number: A0A0A7KJM4; SEQ ID NO: 23).

The gene encoding the phytoene desaturase in the recombinant Deinococcus bacterium of the invention may be easily identified using routine methods, for example based on homology with the nucleic acid encoding any of the above identified phytoene desaturases.

To enhance the production of phytoene, the recombinant bacterium of the invention may also be genetically modified in order to increase the production of geranylgeranyl diphosphate (GGPP).

In particular, the recombinant bacterium of the invention may be genetically modified to increase the carbon flux to isopentenyl pyrophosphate (IPP) and dimethylallyl pyrophosphate (DMAPP) and/or to increase the conversion of IPP and DMAPP to geranylgeranyl diphosphate (GGPP).

The carbon flux to IPP and DMAPP may be increased by enhancing the 2-C-methyl-D-erythritol 4-phosphate/1-deoxy-D-xylulose 5-phosphate (MEP/DXP) pathway. As used herein, the term “MEP pathway” or “MEP/DXP pathway” refers to the biosynthetic pathway leading to the formation of IPP and DMAPP from the condensation of pyruvate and D-glyceraldehyde 3-phosphate to 1-deoxy-D-xylulose 5-phosphate (DXP). This pathway involves the following enzymes: 1-deoxy-D-xylulose 5-phosphate synthase (EC 2.2.1.7), 1-deoxy-D-xylulose 5-phosphate reductoisomerase (EC 1.1.1.267), 2-C-methyl-D-erythritol 4-phosphate cytidylyltransferase (EC 2.7.7.60), 4-diphosphocytidyl-2-C-methyl-D-erythritol kinase (EC 2.7.1.148), 2-C-methyl-D-erythritol 2,4-cyclodiphosphate synthase (EC 4.6.1.12), 4-hydroxy-3-methylbut-2-en-1-yl diphosphate synthase (EC 1.17.7.1), 4-hydroxy-3-methylbut-2-enyl diphosphate reductase (EC 1.17.1.2), and isopentenyl-diphosphate delta-isomerase (EC 5.3.3.2).

This pathway may be enhanced by any method known by the skilled person, for example by a method described in the patent application WO 2015/189428. In particular, this pathway may be enhanced by increasing at least one enzymatic activity selected from the group consisting of DXP synthase (DXS), DXP reductoisomerase (DXR), IspD, IspE, IspF, IspG, IspH and IPP isomerase activities (IDI), preferably by increasing at least DXP synthase and IPP isomerase activities.

An enzymatic activity (e.g. DXS, DXR, IspD, IspE, IspF, IspG, IspH, IDI or FPPS activity) may be increased as detailed above for the phytoene synthase activity, i.e. by verexpression of an endogenous gene or expression of a heterologous gene, and/or expression of an improved variant of the endogenous enzyme.

The term “DXS” or “DXP synthase” refers to the enzyme 1-deoxy-D-xylulose 5-phosphate synthase (EC 2.2.1.7) encoded by the dxs gene which catalyzes the condensation of pyruvate and D-glyceraldehyde 3-phosphate to 1-deoxy-D-xylulose 5-phosphate (DXP). The names of gene product, “DXP synthase”, “DXS” or “DXPS”, are used interchangeably in this application. The DXP synthase activity can be determined by using a radiolabelled substrate as described by Lois et al. (1998) or any other method known by the skilled person. The term “DXP reductoisomerase” or “DXR” refers to the enzyme 1-deoxy-D-xylulose 5-phosphate reductoisomerase (EC 1.1.1.267) encoded by the dxr gene. The term “IspD” refers to the enzyme 2-C-methyl-D-erythritol 4-phosphate cytidylyltransferase (EC 2.7.7.60) encoded by the ispD gene. The term “IspE” refers to the enzyme 4-diphosphocytidyl-2-C-methyl-D-erythritol kinase, (EC 2.7.1.148) encoded by the ispE gene. The term “IspF” refers to the enzyme 2-C-methyl-D-erythritol 2,4-cyclodiphosphate synthase (EC 4.6.1.12) encoded by the ispF gene. The term “IspG” refers to the enzyme 4-hydroxy-3-methylbut-2-en-1-yl diphosphate synthase (EC 1.17.7.1) encoded by the ispG gene. The term “IspH” refers to the enzyme 4-hydroxy-3-methylbut-2-enyl diphosphate reductase, also named hydroxymethylbutenyl pyrophosphate reductase, (EC 1.17.1.2) encoded by the ispH gene. The term “IDI”, “IPP isomerase” or “isopentenyl pyrophosphate isomerase” refers to the enzyme isopentenyl-diphosphate delta-isomerase (EC 5.3.3.2) encoded by the idi gene that catalyzes the 1,3-allylic rearrangement of the homoallylic substrate isopentenyl (IPP) to its allylic isomer, dimethylallyl diphosphate (DMAPP). According to the organism, the nomenclature of the above identified enzymes and encoding genes may vary. However, for the sake of clarity, in the present specification, these terms are used independently from the origin of the enzymes or genes.

Preferably, at least one gene selected from the group consisting of dxs, dxr, ispD, ispE, ispF, ispG, ispH and idi genes, is overexpressed, more preferably at least dxs and/or idi genes, and even more preferably at least dxs and idi genes are overexpressed. These genes may be endogenous or heterologous, preferably endogenous. dxs, dxr, ispD, ispE, ispF, ispG, ispH and idi genes of the recombinant Deinococcus bacterium of the invention may be easily identified as described in the patent application WO 2015/189428.

In a particular embodiment, the recombinant Deinococcus bacterium of the invention is genetically modified to overexpress an endogenous dxs gene or to express a heterologous dxs gene, and to overexpress an endogenous idi gene or to express a heterologous idi gene.

In a preferred embodiment, the overexpressed endogenous or expressed heterologous dxs gene is from a Deinococcus bacterium. Examples of dxs genes from Deinococcus bacteria include, but are not limited to, the dxs genes from D. geothermalis (SEQ ID NO: 24; UniProt accession number: Q11ZP0), D. yunweiensis (SEQ ID NO: 25), D. deserti (NCBI Accession number: WP_012692944.1; GenBank: AC045821.1; UniProt accession number: C1D1U7), D. radiodurans (UniProt accession number: Q9RUB5; NCBI Accession number: WP_010888114.1) and D. radiopugnans (SEQ ID NO: 26). Preferably, the dxs gene is selected from the group consisting of the dxs genes from D. geothermalis, D. yunweiensis and D. radiopugnans. More preferably, the dxs gene is from D. yunweiensis or D. radiopugnans. Any polypeptide, preferably from a Deinococcus bacterium, having at least 70%, preferably 80%, more preferably 90%, sequence identity to any of the polypeptides encoded by those genes, preferably to the polypeptide encoded by SEQ ID NO: 24, 25 or 26, and having a DXS activity may also be used in the present invention.

In a preferred embodiment, the overexpressed endogenous or expressed heterologous idi gene is from a Deinococcus bacterium. Examples of idi genes from Deinococcus bacteria include, but are not limited to, the idi genes from D. geothermalis (SEQ ID NO: 27), D. yunweiensis (SEQ ID NO: 28), D. deserti (NCBI Accession number: WP_012692934.1), D. radiodurans (UniProt accession number: Q9RVE2.3) or D. radiopugnans (SEQ ID NO: 29). Preferably, the idi gene is selected from the group onsisting of the idi genes from D. geothermalis and D. yunweiensis. More preferably, the idi gene is from D. yunweiensis. Any polypeptide, preferably from a Deinococcus bacterium, having at least 70%, preferably 80%, more preferably 90%, sequence identity to any of the polypeptides encoded by those genes, preferably to the polypeptide encoded by SEQ ID NO: 27, 28 or 29, and having an IDI activity may also be used in the present invention.

In addition, or alternatively, the recombinant bacterium of the invention may also express a variant of a Deinococcus DXP synthase which exhibits increased activity by comparison to the wild-type enzyme. Such improved DXP synthases are described in the international patent applications WO 2015/189428 and WO 2012/052171.

In particular, the recombinant bacterium of the invention may express a gene encoding R244C mutant of the DXP synthase from D. radiopugnans (SEQ ID NO: 30), a gene encoding R238C mutant of the DXP synthase from D. yunweiensis (SEQ ID NO: 31) and/or a gene encoding R241C mutant of the DXP synthase from D. geothermalis (SEQ ID NO: 32).

In addition, or alternatively, the phytoene production may also be improved by increasing the conversion of IPP and DMAPP to GGPP. Preferably, in the recombinant bacterium of the invention, the FPP synthase activity is increased by comparison to the wild-type bacterium.

As used herein, the term “IspA”,“FDPS”,“FPPS” or “FPP synthase” refers to an enzyme encoded by the fdps (or crtE) gene and exhibiting farnesyl diphosphate synthase activity (EC 2.5.1.10), dimethylallyltranstransferase activity (EC 2.5.1.1) and geranylgeranyl diphosphate synthase activity (EC 2.5.1.29).

Preferably, the FPP synthase activity is increased by overexpression of an endogenous gene or expression of a heterologous fdps gene. In particular, the recombinant Deinococcus bacterium of the invention may comprise a heterologous nucleic acid encoding a polypeptide exhibiting FPP synthase activity and/or may overexpress an endogenous nucleic acid encoding a polypeptide exhibiting FPP synthase activity.

The polypeptide exhibiting FPP synthase activity may be any known FPP synthase, preferably any FPP synthase from Deinococcus bacteria.

Examples of Deinococcus FPP synthases include, but are not limited to, FPP synthases from D. geothermalis (NCBI Accession number: ABF45913; SEQ ID NO: 33), D. radiodurans (NCBI Accession number: NP_295118; SEQ ID NO: 34) and D. deserti (NCBI Accession number: AC046371; SEQ ID NO: 35). The polypeptide exhibiting FPP synthase activity may also be any polypeptide exhibiting FPP synthase activity and having at least 60%, preferably 65, 70, 75, 80, 85, 90, 95, 96, 97, 98 or 99%, identity to any FPP synthase listed above.

In a particular embodiment, the polypeptide exhibiting FPP synthase activity is selected from the group consisting of

a) a polypeptide comprising, or consisting of, an amino acid sequence selected from the group consisting of SEQ ID NO: 33 to 35; and

b) a polypeptide exhibiting FPP synthase activity and having an amino acid sequence having at least 60%, preferably 65, 70, 75, 80, 85, 90, 95, 96, 97, 98 or 99%, identity to SEQ ID NO: 33, 34 or 35.

In a particular embodiment, the recombinant bacterium of the invention is genetically modified in order to increase the carbon flux to isopentenyl pyrophosphate (IPP) and dimethylallyl pyrophosphate (DMAPP) and to increase the conversion of IPP and DMAPP to geranylgeranyl diphosphate (GGPP).

Preferably, the recombinant bacterium of the invention exhibits increased FPP synthase, DXP synthase and/or IPP isomerase activities by comparison to the wild-type bacterium. More preferably, the recombinant bacterium of the invention exhibits increased FPP synthase, DXP synthase and IPP isomerase activities by comparison to the wild-type bacterium.

In a more particular embodiment, the recombinant bacterium of the invention, preferably a D. geothermalis bacterium, is genetically modified

to exhibit increased phytoene synthase activity, preferably by overexpressing a native CrtB gene,

to exhibit decreased phytoene desaturase activity, preferably by deleting all or part of the Crtl gene encoding a phytoene desaturase,

to exhibit increased FPP synthase activity, preferably by overexpressing a native FPP synthase gene;

to exhibit increased DXP synthase activity, preferably by expressing a variant of a Deinococcus DXP synthase, more preferably the variant encoded by SEQ ID NO: 31, and

to exhibit increased IPP isomerase activity, preferably by overexpressing a native idi gene or express a heterologous idi gene, more preferably expressing the enzyme encoded by SEQ ID NO: 28.

In a preferred embodiment, the recombinant bacterium of the invention, preferably a Deinococcus geothermalis, and more preferably a recombinant bacterium as defined in the previous paragraph, is able to produce at least 5 mg/g DCW of phytoene, preferably at least 10 mg/g DCW of phytoene, more preferably at least 15 mg/g DCW of phytoene, and even more preferably at least 20 mg/g DCW of phytoene, when cultured in aerobiosis and in the presence of glucose as carbon source. Preferably, the recombinant bacterium of the invention produces only one isomer of phytoene, i.e. 15-cis phytoene, and does not produce phytofluene, ζ-carotene, neurosporene or lycopene.

In another aspect, the present invention also relates to a cell extract of the recombinant Deinococcus bacterium of the invention. As used herein, the term “cell extract” refers to any fraction obtained from a host cell, such as a cell supernatant, a cell debris, cell walls, DNA or RNA extract, enzymes or enzyme preparation or any preparation derived from host cells by chemical, physical and/or enzymatic treatment, which is essentially or mainly free of living cells.

In a particular embodiment, the cell extract comprises phytoene and is preferably a fraction comprising cell membranes. In particular, the cell extract may comprise phytoene and lipids, such as membrane lipids.

In a preferred embodiment, the cell extract comprises phytoene and does not comprise any detectable amount of phytofluene, ζ-carotene, neurosporene or lycopene.

In a more particular embodiment, the cell extract comprises only one carotenoid compound, said compound being phytoene.

In preferred embodiments, the extract comprises only one isomer of phytoene, i.e. 15-cis phytoene, and does not comprise phytofluene, ζ-carotene, neurosporene or lycopene.

The invention further relates to the use of said cell extract to produce phytoene.

In a further aspect, the present invention relates to a use of a recombinant Deinococcus bacterium of the invention for producing phytoene.

In particular, the present invention relates to a method of producing phytoene, preferably 15-cis phytoene, comprising (i) culturing a recombinant Deinococcus bacterium according to the invention under conditions suitable to produce phytoene and optionally (ii) recovering said phytoene.

The method may further comprise isolating or purifying said phytoene.

Preferably, the recombinant Deinococcus bacterium does not produce significant or detectable amount of phytofluene or of any other intermediate molecules from phytoene to lycopene.

Thus, in a particular embodiment, phytoene produced by the method of the invention or according to the use of the invention is exempt of phytofluene or of any other intermediate molecules from phytoene to lycopene (i.e. ζ-carotene, neurosporene), or lycopene.

As used herein, the term “exempt of” means that phytoene does not contain any detectable amounts of phytofluene or of any other intermediate molecules from phytoene to lycopene, preferably of phytofluene. The presence of phytofluene or of any other intermediate molecules may be assessed by any method known by the skilled person such as HPLC analysis.

All embodiments described above for the recombinant Deinococcus bacterium of the invention are also contemplated in this aspect.

Conditions suitable to produce phytoene may be easily determined by the skilled person according to the recombinant Deinococcus bacterium used.

In particular, the carbon source may be selected from the group consisting of C5 sugars such as xylose and arabinose, C6 sugars such as glucose, cellobiose, saccharose and starch. In a preferred embodiment, the carbon source is glucose.

Preferably, the recombinant bacterium of the invention is cultured in aerobiosis and in the presence of glucose as carbon source.

Alternatively, phytoene is produced from renewable, biologically derived carbon sources such as cellulosic biomass As used herein, the term “cellulosic biomass” refers to any biomass material, preferably vegetal biomass, comprising cellulose, hemicellulose and/or lignocellulose, preferably comprising cellulose and hemicellulose. Cellulosic biomass includes, but is not limited to, plant material such as forestry products, woody feedstock (softwoods and hardwoods), agricultural wastes and plant residues (such as corn stover, shorghum, sugarcane bagasse, grasses, rice straw, wheat straw, empty fruit bunch from oil palm and date palm, agave bagasse, from tequila industry), perennial grasses (switchgrass, miscanthus, canary grass, erianthus, napier grass, giant reed, and alfalfa); municipal solid waste (MSW), aquatic products such as algae and seaweed, wastepaper, leather, cotton, hemp, natural rubber products, and food processing by-products.

Preferably, if the cellulosic biomass comprises lignocellulose, this biomass is pre-treated before hydrolysis. This pretreatment is intended to open the bundles of ignocelluloses in order to access the polymer chains of cellulose and hemicellulose. Pretreatment methods are well known by the skilled person and may include physical pretreatments (e.g. high pressure steaming, extrusion, pyrolysis or irradiation), physicochemical and chemical pretreatments (e.g. ammonia fiber explosion, treatments with alkaline, acidic, solvent or oxidizing agents) and/or biological pretreatments.

Temperature conditions can also be adapted depending on the use of mesophilic or thermophilic Deinococcus bacteria.

In an embodiment, the Deinococcus bacteria is a thermophilic Deinococcus, such as for example D. geothermalis or D. murrayi, and the culture of the recombinant Deinococcus bacterium under conditions suitable to produce phytoene is performed at a temperature comprised between 30° C. and 55° C., preferably between 35 and 50° C., more preferably between 40° C. and 50° C., and even more preferably between 45 and 48° C.

In another embodiment, the Deinococcus bacteria is a mesophilic Deinococcus, such as for example D. grandis, D. aquaticus, D. indicus, D. cellulosilyticus or D. depolymerans, and the culture of the recombinant Deinococcus bacterium under conditions suitable to produce phytoene is performed at a temperature comprised between 20° C. and 40° C., preferably between 28 and 35° C., more preferably at about 30° C.

In a preferred embodiment, at least 5 mg/g DCW of phytoene, preferably at least mg/g DCW of phytoene, more preferably at least 15 mg/g DCW of phytoene, and even more preferably at least 20 mg/g DCW of phytoene, are produced and/or recovered with the method of the invention.

Optionally, the method may further comprise submitting produced or recovered phytoene to an isomerization step, e.g. using an isomerase, to produce another phytoene isomer.

The methods of the invention may be performed in a reactor, in particular a reactor of conversion of biomass. By “reactor” is meant a conventional fermentation tank or any apparatus or system for biomass conversion, typically selected from bioreactors, biofilters, rotary biological contactors, and other gaseous and/or liquid phase bioreactors. The apparatus which can be used according to the invention can be used continuously or in batch loads. Depending on the cells used, the method may be conducted under aerobiosis, anaerobiosis or microaerobiosis.

The present invention further relates to a reactor comprising a recombinant Deinococcus bacterium of the invention, or a cell extract thereof. Preferably, the reactor further comprises a carbon source, more preferably a biologically derived carbon source such as cellulosic biomass. The invention further relates to the use of said reactor to produce phytoene.

The present invention also relates to a composition comprising a recombinant Deinococcus bacterium of the invention or an extract thereof and the use of said composition to produce phytoene. Preferably, the composition further comprises a carbon source, more preferably a biologically derived carbon source such as cellulosic biomass.

The invention also relates to phytoene, preferably isolated or purified phytoene, obtained by a method of the invention. Isolated phytoene is typically devoid of at least some proteins or other constituents of the cells to which it is normally associated or with which it is normally admixed or in solution. Purified phytoene is typically substantially devoid of other constituents of the cells.

The present invention further relates to a composition comprising phytoene obtained by a method of the invention.

In an embodiment, said composition is a cosmetic or pharmaceutical composition.

In another embodiment, said composition is a nutraceutical or nutricosmetic composition, or a food or feed additive.

As used herein, the term “nutraceutical composition” refers to a composition comprising nutrients isolated or purified from food and having a beneficial effect on the health of the consumer. As used herein, the term “nutricosmetic composition” refers to a composition comprising nutritional oral ingredients and which is formulated and marketed specifically for beauty purposes.

In a particular embodiment, the cosmetic or pharmaceutical composition is a skin whitening, lightening or bleaching composition or a composition to prevent aging, oxidative or photo-oxidative damages.

Phytoene was also known to show anticarcinogenic and anti-inflammatory properties. Thus, in an embodiment, the composition is a pharmaceutical composition to be used in the treatment of cancer or in the treatment of inflammatory disorders.

Preferably, the composition of the invention is to be administered by topical or oral route.

Preferably, said composition does not comprise any detectable amount of phytofluene, ζ-carotene, neurosporene and/or lycopene. More preferably, said composition does not comprise any detectable amount of phytofluene.

Preferably, the composition comprises only one isomer of phytoene which is 15-cis phytoene.

The composition may further comprise a recombinant Deinococcus bacterium of the invention or an extract thereof.

The composition of the invention may obviously, depending on its use, comprise also other ingredients, such as cosmetic or pharmaceutical acceptable carriers, preservatives, antioxidants such as carotenoids, as well as pharmaceutically or cosmetically active ingredients.

In a preferred embodiment, the composition further comprises a hydrophobic carrier, which may be selected from oils typically used in the cosmetic, pharmaceutical or food industry, such as vegetable, mineral or synthetic oils.

The present invention also relates to a method of producing a composition of the invention, in particular a cosmetic, pharmaceutical, nutraceutical, nutricosmetic composition or a food or feed additive, comprising (i) culturing a recombinant Deinococcus bacterium according to the invention under conditions suitable to produce phytoene, (ii) recovering said phytoene, and (iii) mixing said phytoene with at least one carrier or at least one other ingredient of the cosmetic, pharmaceutical, nutraceutical, nutricosmetic composition or food or feed additive.

All embodiments described above for the recombinant Deinococcus bacterium, the method of producing phytoene and the composition of the invention are also contemplated in this aspect.

Further aspects and advantages of the present invention will be described in the following examples, which should be regarded as illustrative and not limiting.

### EXAMPLES

### Example 1

A Deinococcus geothermalis strain was genetically engineered to produce phytoene. The recombinant D. geothermalis producing phytoene was obtained by disrupting a part of the carotenoid pathway, i.e. the phytoene desaturase (E.C. 1.3.99.26, 1.3.99.28, 1.3.99.29, 1.3.99.31) (crtl) gene was knockout. The resulting constructs were checked by sequencing.

To make seed cultures, individual colonies were picked to inoculate 25 ml of CMG2% medium (Peptone 2 g/L; Yeast Extract 5 g/L; Glucose 55 mM (20 g/L); MOPS acid 40 mM; NH₄Cl 20 mM; NaOH 10 mM; KOH 10 mM; CaCl₂.2H₂O 0.5 μM; Na₂SO₄.10H₂O 0.276 mM; MgCl₂.6H₂O 0.528 mM; (NH₄)₆(Mo₇)O₂₄.4H₂O 3 nM; H₃BO₃ 0.4 μM; CoCl₂.6H₂O 30 nM; CuSO₄.5H₂O 10 nM; MnCl₂ 0.25 μM; ZnSO₄.7H₂O 10 nM; D-Biotin 1 μg/L; Niacin (nicotinic acid) 1 μg/L; B6 vitamin 1 μg/L; B1 vitamin; FeCl₃ 20 μM; Sodium Citrate.2H₂O 20 μM; K₂HPO₄ 5.7 mM) containing 2% glucose or dextrose as the main carbon source, and cultured at 45° C. and 250 rpm overnight. Seed from log phase of growth was then inoculated into 25 ml of the same fresh medium at an initial optical density at 600 nm (OD600) of 0.4. This second seed culture was cultured at 45° C. and 250 rpm overnight. The cultures for phytoene production were performed at 45° C. and 250 rpm for 24 h from log phase of growth inoculated into 25 ml of mineral defined medium (NH₄)₂SO₄<100 mM; NaH₂PO₄.H₂O<10 mM; KCl<10 mM; Na₂SO₄<10 mM; Acide citrique<30 mM; MgCl₂.6H₂O<10 mM; CaCl₂.2H₂O<10 mM; ZnCl₂<50 mg/L; FeSO₄.7H2O<50 mg/L; MnCl₂.4H₂O<50 mg/L; CuSO₄<50 mg/L; CoCl₂.6H₂O<50 mg/L; H₃BO₃<5 mg/L; MES<200 mM; (NH₄)6Mo₇O₂₄.4H₂O<0.5 mM; Glucose or dextrose<30 g/L (166 mM) at an initial optical density at 600 nm (OD600) of 0.4.

After 24 h of culture, 1 mL of culture was centrifuged and carotenoid extraction was done by mixing 1 mL of ethanol with pellet. The ethanol phase was analyzed by absorbance at OD 285 nm and analyzed by HPLC (Column c18 poroshell agilent 150 mm*2.1 mm*2.7 um, mobile phase acetonitrile/methanol/ethyl acetate).

Finally, phytoene yield in mg/g of dry cell weight (DCW) was determined by the dilution of phytoene standard. The recombinant D. geothermalis produced 0.4 mg/g DCW of phytoene and did not produce any detectable amount of phytofluene, ζ-carotene, neurosporene or lycopene.

The phytoene isomer produced by the recombinant D. geothermalis was analyzed by RMN and identified as 15-cis phytoene.

### Example 2

The recombinant Deinococcus geothermalis strain of example 1 was further modified by inserting into the chromosome an expression cassette comprising

(i) a gene encoding the R238C mutant of 1-deoxy-D-xylulose 5-phosphate synthase (E.C. 2.2.1.7) (DXS) from Deinococcus yunweinensis, and

(ii) a gene encoding the isopentenyl-diphosphate delta-isomerase (EC 5.3.3.2) (IDI) from Deinococcus yunweinensis.

These genes were placed under the control of a constitutive promoter and the expression cassette was inserted into the chromosome replacing the amylase (amy) gene. The resulting constructs were checked by sequencing.

Seed cultures and cultures for the production of phytoene were carried out as described in example 1.

After 24h of culture, 1 mL of culture was centrifuged and carotenoid extraction was done by mixing 1 mL of ethanol with pellet. The ethanol phase was analyzed by absorbance at OD 285 nm and analyzed by HPLC (Column c18 poroshell agilent 150 mm*2.1 mm*2.7 um, mobile phase acetonitrile/methanol/ethyl acetate).

It was thus determined that the recombinant D. geothermalis produced about 1 mg/g DCW (dry cell weight) of phytoene and did not produce any detectable amount of phytofluene, ζ-carotene, neurosporene or lycopene.

### Example 3

The recombinant Deinococcus geothermalis strain of example 1 was further modified by inserting into the chromosome an expression cassette comprising

(i) a gene encoding the R238C mutant of 1-deoxy-D-xylulose 5-phosphate synthase (E.C. 2.2.1.7) (DXS) from Deinococcus yunweinensis,

(ii) a gene encoding the isopentenyl-diphosphate delta-isomerase (EC 5.3.3.2) (IDI) from Deinococcus yunweinensis,

(iii) a gene encoding the farnesyl pyrophosphate synthase (E.C. 2.5.1.1, 2.5.1.10, 2.5.1.29) (FPPS) from Deinococcus geothermalis, and

(iv) a gene encoding the phytoene synthase (EC 2.5.1.32) (CrtB) from Deinococcus geothermalis.

These genes were placed under the control of a constitutive promoter and the expression cassette was inserted into the chromosome replacing the amylase (amy) gene. The resulting constructs were checked by sequencing.

Seed cultures and cultures for the production of phytoene were carried out as described in example 1.

After 24 h of culture, 1 mL of culture was centrifuged and carotenoid extraction was done by mixing 1 mL of ethanol with pellet. The ethanol phase was analyzed by absorbance at OD 285 nm and analyzed by HPLC (Column c18 poroshell agilent 150 mm*2.1 mm*2.7 um, mobile phase acetonitrile/methanol/ethyl acetate).

It was thus determined that the recombinant D. geothermalis produced about 22 mg/g DCW (dry cell weight) of phytoene and did not produce any detectable amount of phytofluene, ζ-carotene, neurosporene or lycopene.

### Example 4

The recombinant Deinococcus geothermalis strain of example 1 was further modified by inserting into the chromosome a first expression cassette comprising

(i) a gene encoding the R238C mutant of 1-deoxy-D-xylulose 5-phosphate synthase (E.C. 2.2.1.7) (DXS) from Deinococcus yunweinensis, and

(ii) a gene encoding the isopentenyl-diphosphate delta-isomerase (EC 5.3.3.2) (IDI) from Deinococcus yunweinensis,

and a second expression cassette comprising

(iii) a gene encoding the farnesyl pyrophosphate synthase (E.C. 2.5.1.1, 2.5.1.10, 2.5.1.29) (FPPS) from Deinococcus geothermalis, and

(iv) a gene encoding the phytoene synthase (EC 2.5.1.32) (CrtB) from Deinococcus geothermalis.

These genes were placed under the control of constitutive promoters. The first and second expression cassettes were inserted into the chromosome replacing the amylase (amy) gene and the endogenous fdps gene, respectively. The resulting constructs were checked by sequencing.

Seed cultures and cultures for the production of phytoene were carried out as described in example 1.

After 24 h of culture, 1 mL of culture was centrifuged and carotenoid extraction was done by mixing 1 mL of ethanol with pellet. The ethanol phase was analyzed by absorbance at OD 285 nm and analyzed by HPLC (Column c18 poroshell agilent 150 mm*2.1 mm*2.7 um, mobile phase acetonitrile/methanol/ethyl acetate).

It was thus determined that the recombinant D. geothermalis produced 23 mg/g DCW (dry cell weight) of phytoene and did not produce any detectable amount of phytofluene, ζ-carotene, neurosporene or lycopene.

### Example 5

The recombinant Deinococcus geothermalis strain of example 1 is further modified by inserting into the chromosome an expression cassette comprising

(i) a gene encoding the 1-deoxy-D-xylulose 5-phosphate synthase (E.C. 2.2.1.7) (DXS) from Deinococcus yunweinensis or Deinococcus geothermalis,

(ii) a gene encoding the isopentenyl-diphosphate delta-isomerase (EC 5.3.3.2) (IDI) from Deinococcus yunweinensis,

(iii) a gene encoding the farnesyl pyrophosphate synthase (E.C. 2.5.1.1, 2.5.1.10, 2.5.1.29) (FPPS) from Deinococcus geothermalis, and

(iv) a gene encoding the phytoene synthase (EC 2.5.1.32) (CrtB) from Deinococcus geothermalis.

These genes are placed under the control of a constitutive promoter and the expression cassette is inserted into the chromosome replacing the amylase (amy) gene. The resulting constructs are checked by sequencing.

Seed cultures and cultures for the production of phytoene are carried out as described in example 1. After 24 h of culture, 1 mL of culture is centrifuged and carotenoid extraction is done by mixing 1 mL of ethanol with pellet. The ethanol phase is analyzed by absorbance at OD 285 nm and analyzed by HPLC (Column c18 poroshell agilent 150 mm*2.1 mm*2.7 um, mobile phase acetonitrile/methanol/ethyl acetate).

### Example 6

The recombinant Deinococcus geothermalis strain of example 1 is further modified by inserting into the chromosome a first expression cassette comprising

(i) a gene encoding the 1-deoxy-D-xylulose 5-phosphate synthase (E.C. 2.2.1.7) (DXS) from Deinococcus yunweinensis or Deinococcus geothermalis, and

(ii) a gene encoding the isopentenyl-diphosphate delta-isomerase (EC 5.3.3.2) (IDI) from Deinococcus yunweinensis,

and a second expression cassette comprising

(iii) a gene encoding the farnesyl pyrophosphate synthase (E.C. 2.5.1.1, 2.5.1.10, 2.5.1.29) (FPPS) from Deinococcus geothermalis, and

(iv) a gene encoding the phytoene synthase (EC 2.5.1.32) (CrtB) from Deinococcus geothermalis.

These genes are placed under the control of constitutive promoters. The first and second expression cassettes are inserted into the chromosome replacing the amylase (amy) gene and the endogenous fdps gene, respectively. The resulting constructs are checked by sequencing.

Seed cultures and cultures for the production of phytoene are carried out as described in example 1. After 24 h of culture, 1 mL of culture is centrifuged and carotenoid extraction is done by mixing 1 mL of ethanol with pellet. The ethanol phase is analyzed by absorbance at OD 285 nm and analyzed by HPLC (Column c18 poroshell agilent 150 mm*2.1 mm*2.7um, mobile phase acetonitrile/methanol/ethyl acetate).

1-23. (canceled)

24. A method of producing phytoene comprising culturing a recombinant Deinococcus bacterium under conditions suitable to produce phytoene, and optionally recovering said phytoene, wherein said recombinant Deinococcus bacterium is genetically modified to exhibit increased phytoene synthase activity and decreased phytoene desaturase activity.

25. The method of claim 24, wherein the recombinant Deinococcus bacterium does not exhibit any phytoene desaturase activity.

26. The method of claim 24, wherein, the recombinant Deinococcus bacterium is genetically modified by inactivating a gene encoding a phytoene desaturase by deleting all or part of said gene or introducing a nonsense codon, a cassette, a gene or a mutation inducing a frameshift.

27. The method of claim 26, wherein the recombinant Deinococcus bacterium is genetically modified by deleting all or part of a gene encoding a phytoene desaturase.

28. The method of claim 24, wherein the recombinant Deinococcus bacterium is genetically modified to overexpress a native gene encoding phytoene synthase.

29. The method of claim 24, wherein the recombinant Deinococcus bacterium is genetically modified to express a heterologous gene encoding phytoene synthase or a native gene encoding phytoene synthase and comprising a mutation improving phytoene synthase activity of the encoded enzyme.

30. The method of claim 24, wherein the recombinant Deinococcus bacterium is genetically modified to express a gene encoding a feedback resistant phytoene synthase.

31. The method of claim 24, wherein the recombinant Deinococcus bacterium further exhibits increased FPP synthase activity.

32. The method of claim 24, wherein the recombinant Deinococcus bacterium further exhibits increased DXP synthase and/or IPP isomerase activities.

33. The method of claim 24, wherein the recombinant Deinococcus bacterium is a Deinococcus bacterium selected from D. geothermalis, D. murrayi, D. grandis, D. aquaticus, D. indicus, D. cellulosilyticus, D. depolymerans, or D. maricopensis.

34. The method of claim 24, wherein the recombinant Deinococcus bacterium is a Deinococcus geothermalis bacterium.

35. The method of claim 24, wherein the recombinant Deinococcus bacterium is able to produce at least 20 mg/g DCW (dry cell weight) of phytoene.

36. The method of claim 35, wherein the recombinant Deinococcus bacterium is able to produce at least 20 mg/g DCW (dry cell weight) of phytoene when cultured in aerobiosis and in the presence of glucose as carbon source.

37. The method of claim 24, wherein the recombinant Deinococcus bacterium produces only one isomer of phytoene which is 15-cis phytoene, and does not produce phytofluene, ζ-carotene, neurosporene or lycopene.

38. The method of claim 24, wherein the recombinant Deinococcus bacterium is a thermophilic Deinococcus and the culture of the recombinant Deinococcus bacterium under conditions suitable to produce phytoene is performed at a temperature between 40 and 50° C.

39. A recombinant Deinococcus bacterium genetically modified to exhibit increased phytoene synthase activity and decreased phytoene desaturase activity.

40. A cell extract of the recombinant Deinococcus bacterium of claim 39, wherein said cell extract comprises phytoene and does not comprise any detectable amount of phytofluene, ζ-carotene, neurosporene or lycopene.

41. The cell extract of claim 40, which comprises only one isomer of phytoene which is 15-cis phytoene.

42. The cell extract of claim 40, said fraction comprising cell membranes.

43. A composition comprising phytoene obtained by the method of claim 24, wherein said composition does not comprise any detectable amount of phytofluene, ζ-carotene, neurosporene or lycopene.

44. The composition of claim 43, said composition comprising only one isomer of phytoene which is 15-cis phytoene.

45. The composition of claim 43, said composition being a cosmetic composition, pharmaceutical composition, nutraceutical composition, nutricosmetic composition, a food or a feed additive.


SEQUENCE LISTING (35 sequences)

SEQ ID NO: 1 - protein, 296 aa, Pantoea agglomerans
MEVGSKSFATASKLFDAKTRRSVLMLYAWCRHCDDVIDDQVLGFSNDTPSLQSAEQRLAQLEMKTRQAYAGSQMHEPAFAAFQEVAMAHDILPAYAFDHLAGFAMDVHETRYQTLDDTLRYCYHVAGVVGLMMAQIMGVRDNATLDRACDLGLAFQLTNIARDIVEDAEAGRCYLPAAWLAEEGLTRENLADPQNRKALSRVARRLVETAEPYYRSASAGLPGLPLRSAWAIATAQQVYRKIGMKVVQAGSQAWEQRQSTSTPEKLALLVAASGQAVTSRVARHAPRSADLWQRPV

SEQ ID NO: 2 - protein, 304 aa, Paracoccus sp_N81106
MSDLVLTSTEAITQGSQSFATAAKLMPPGIRDDTVMLYAWCRHADDVIDGQALGSRPEAVNDPQARLDGLRADTLAALQGDGPVTPPFAALRAVARRHDFPQAWPMDLIEGFAMDVEARDYRTLDDVLEYSYHVAGIVGVMMARVMGVRDDPVLDRACDLGLAFQLTNIARDVIDDARIGRCYLPGDWLDQAGARVDGPVPSPELYTVILRLLDAAELYYASARVGLADLPPRCAWSIAAALRIYRAIGLRIRKGGPEAYRQRISTSKAAKIGLLGIGGWDVARSRLPGAGVSRQGLWTRPHHA

SEQ ID NO: 3 - protein, 298 aa, Deinococcus geothermalis
MPYPAFPSPTDSRPPAAAARHCWEVTRQHSRTFSLGSRFFPAGQREAVWAVYAACRAGDDSVDECHGNEAKQALAAWWARVQAAFAGRPGPHPIDTALAWAVSHWPIPLAAFEELHEGLRMDLEGYHYRDLEDLTLYCRRVAGVVGFMIAPICGYSGGEDTLRRALRLGQAMQLTNILRDVGEDLERGRVYLPEDLLREYGVTRAALEEGRVTPGYRALMRDLSALARAWYAEGRAGIPCLRGRARIGVQAAARAYEGILDDLARGDFDNFRRRAHVSGPRKLLMLPQAWWEVRGAPP

SEQ ID NO: 4 - protein, 297 aa, Deinococcus actinosclerus
MTFTSASPPGLDRAVAHCQDVTREHSKTFYLGSRFFPAAQRRAVWAVYAACRDGDDTVDELSGHAAQLGLDQWWTRVQGAFAGRPGDHPIDMALAWAAREYPIPLSAFAELHEGLRMDLSGHEYHSMADLILYCRRVAGVVGFMIAPVSGYSGGERTLHAALMLGQAMQLTNILRDVGEDLTRRRVYLPSELLAEFRVSRADLERGVVTPEYRALMRHLSALAREWYAEGRQGIPCLHGSARLAVATAARAYEGILDDLARNDFDNFGRRAHVSGPRKLMMLPQAWWELRTAPAPLT

SEQ ID NO: 5 - protein, 313 aa, Deinococcus deserti
MPDLNLPGSPPLAALRWCRVMTRVHSQTFYLGSLLYPRRQRLAVWAVYAACRVGDDIADEYTGAGARVELDRWWARVCSAFCGNPGDDPMQTALAWATREYPIPLDAFAELYEGFCMDLTGQSYDSLDDLTLYCRRVAGVVGFMVAPIGGYRGGAHTLEQALMLGQAMQLTNILRDVGEDLSRGRVYLPAELLSRFGVTHADLQAGRVTPEYRALMAQLCGLAREWYARGREGIPALEGRARVGVAAAARTYEGILDDLEAHGYDNFSRRAHVPPRHKLRLLWQEYRAHSSPASACPVAWAEQQYLRLKGLRG

SEQ ID NO: 6 - protein, 273 aa, Deinococcus gobiensis
MTREHSKTFYLGSRCFPGRQRAAVWAVYAACREGDDIADGGGPDVDARLGDWWSRVQGAFAGRPGEHPTDRALAWAAREYPIPLGAFAELHEGLRMDLRGHNYASMDDLTLYCRRVAGVVGFMIAPISGYEGGEATLDKALRLGQAMQLTNILRDVGEDLSLGRVYLPAEVLDRYGLCRADLERGVVTPEYCAMLRDLTAQARAWYAEGRAGIPLLRGRARLAVATAARAYEGILDDLEAAGYDNFNRRAYVSGRRKLMMLPQAWWELRSFSA

SEQ ID NO: 7 - protein, 287 aa, Deinococcus maricopensis
MREEHLPARAIERCRDQTRAHSKTFYFGSRFFPRAQRQAVWAVYAACREGDDIADESPAWDRAAHLDAWWARVRRALNGEGSGDHVSDALAWAARTYPIPESAFEELYEGLRMDLAGHAYDTAEDLELYCRRVAGVVGFMIAPISGFDGGDATLQHALRLGQAMQLTNILRDVGEDWARERVYLPQTLLEQYGVTRTMLARGTVTPEYRALMRHLCAQARTWYAQGRAGIPRLHGGGRLAVAAAARAYEGILDALERNDYDNFNARAHVSGTRKLLLLPQVWWECRA

SEQ ID NO: 8 - protein, 294 aa, Deinococcus peraridilitoris
MAPSDLTAKLPGSAITHCRDVTRDHSKTFYFGSRFFGPAERQAVWAVYAVCRHGDDIVDEGDPHSAPRRLEAWWHGVQGAFAGTPSADPVFQALCWAVARFPIPRGAFEELHLGLRMDLDGHHYRDMTELELYCRRVAGVVGFMIAPIAGFDGGEATLQRALKLGQAMQLTNILRDVGEDAARGRLYLPEDLLSAYRLRARDIHGATVSSEYQALMRHLVATARTWYAEGRSGIPRLRGRARLAVGAAASAYEGILDALEQNDFDNFSRRAQVSGTRKLLMLPGVLWRSRGAPG

SEQ ID NO: 9 - protein, 287 aa, Deinococcus puniceus
MAHCRDVTRLHSKTFYLGSRFFPAQERAAVWAVYAACRDGDDIADELTGHDAVTQLDGWWARMQVAFAGLPDPQPDGTHPIDTALSWAAQTYPIPLSAFGELHEGLRMDLNGHSYQTMDDLTLYCRRVAGVVGFMIAPISGYSGGERTLQHALMLGQAMQLTNILRDVGEDLERGRVYLPADLMTEYGVSGADLQRGIVTPEYRALMRHLSALARDWYKQGRTGIPMLHGSARLAVQTAARAYEGILDDLERADFDNFRRRAYVSGPRKLLMLPQAWWELRGFMARA

SEQ ID NO: 10 - protein, 325 aa, Deinococcus radiodurans
MRSRAGLSLRLPTRTLTVTDYSPALPCTELRRPPLAQAVRYCRDLTRQHSKTFYLGSQLFSPPERAAVWAVYAACRAGDDIVDEAGNGDRERELREWRSRIDAAFAGQPADDPISTALAWAAGRYAIPHSAFAELHEGLNMDLRGHEYRDMDDLLLYCRRVAGVVGFMVAPISGYRGGAATLNDALQLGQAMQLTNILRDVGEDLTRGRVYLPQSLLDEYGLSRAALERWGQGEPLSPAYRALMTHLGGLAREWYAAGRAGIPQLDGRGPLAVLTAARAYEGILDDLERAGYDNFGRRAYVSGRRKLLMLPQAWWELRSLGAVHG

SEQ ID NO: 11 - protein, 283 aa, Deinococcus soli
MAHCQDVTREHSKTFYLGSRFFPMAQRRAVWAVYAACRDGDDTVDELGGTAAQLGLDLWWARVQGAFAGRPGDHPIDMALAWAAREYPIPLSAFEELHEGLRMDLRGHEYHSMDDLILYCRRVAGVVGFMIAPVSGYSGGERTLHAALMLGQAMQLTNILRDVGEDLTRGRVYLPSELLAEFHVSRADLERGVVTPEYRALMRHLSALAREWYAEGRQGIPCLHGSARLAVATAARAYEGILDDLARNDFDNFGRRAHVSGPRKLLMLPQAWWELRTAAASLS

SEQ ID NO: 12 - protein, 299 aa, Deinococcus swuensis
MINTSLPPSPTSAAPVQAVAHCRDVTRDHSKTFFLGSRLFPLRQRQAVWAVYAACRTGDDIADESTADSVEAELDVWWTRIQGAFAGRPGPDPVDTALAWAARTYPIPLSAFAELHEGLRMDLNGHVYHDMEDLALYCRRVAGVIGFMIAPVSGYSGGERTLHHALMLGQAMQLTNILRDVGEDLTRGRVYLPQTLLGEYGVSRADLERGVVTPEYRALMVHLCALARDWYAEGRAGIPCLHGSARLAVATAARAYEGILDDLERADYDNFNRRAYVSGTRKLLMLPRAWWELRGAVSG

SEQ ID NO: 13 - protein, 556 aa, Deinococcus geothermalis
MTDFSTLHRRKQALIIGSGIGGLSLGIRLQSLGFETTILEKLDGPGGRAYQKRTPDGYVFDMGPTVITVPHFIEELFALERDGGWPGQPDYPAEVLNGERVRVGESGGPRTREYVRLVPILPFYRIYFDDGTFFDYDGDPASTRRQIAELAPGDLAGYERFHADAEAIFRRGFLELGYTHFGDVGTMLRVVPDLLKLDAVRTLFSFTSRYFQSPKLRQVFSFETLLIGGNPLSVPAIYAMIHFVEKTWGIHYALGGTGALVRALVRKFEELGGRIEYGAEVEQILVTDEWGKPVRRPLGPRVARGVRLKGGEQRPADLVISNGDWANTYLKRVPRQARLVNNDLRVKAARQSMSLLVIYFGFREDGRSLDLRHHNIILGPRYEALLQEIFGQKVLGADFSQYLHVPTLTDPSLAPPGHHAAYTLIPVPHNGSGLDWTVEGPRLVDRVLAFLDERGYIPGLRERLTYLDFITPDYFEGTLDSYLGNAFGPEPILAQSAYFRPHNRSEDVQNLYLVGAGVQPGAGTPSVMMSAKMTARLIAEDFGLQGAVRPVDSVRR

SEQ ID NO: 14 - protein, 568 aa, Deinococcus actinosclerus
MTPDRTTTPAAARRKTALIVGAGIGGLSLGIRLQSLGFDTTIVERLDQPGGRAYQKRTADGYVFDMGPTVITVPHFIEELFALERDKGMLGEADYPAQVLAPDARVREGESGGERTRDYVKLVPILPFYRIYFDDGTFFDYDGDPVSTRRQIADLAPEDLAGYERFHADAQAIFERGFLELGYTHFGDMPTMLRVVPDLMRLDAVRTLFSFTSKYFSNPKMRQVFSFETLLVGGNPLSVPAIYAMIHFVEKTWGIHYAMGGTGALVDAFARKFEELGGTLRLNAGVQEILVTDDRGRPVRRPGGKRVARGLRLESGEELHADIVVSNGDWANTYLKRVPAAARLVNSDVRVKAARQSMSLLVIYFGFRQEGPALNLRHHNIILGPRYEELLTEIFGKKVLGRDFSQYLHVPTLTDPTLAPEGHHAAYTLVPVPHNASGLDWNVEGPKLVERVYDFLEERGYIPNLRARLTHSEFITPDYFEGTLDSYLGNAFGPEPLLAQSAYFRPHNRSEDVRNLYMVGAGAQPGGGTPSVMMSAKMTARLIAQDFGIHPSVRDGVPERNTAELAAD

SEQ ID NO: 15 - protein, 550 aa, Deinococcus deserti
MARPPELTRRKVAVIIGAGIGGLSLGIRLQSLGFDTTILERLDGPGGRGYQKRTPDGYVFDMGPTVITVPHFIEELFALERDRAALGAEDFPPAVRQAPRVTSGESGGPRTRDYVKLVPILPFYRIVFDDGSYFDYDGDPEGTRRQIAALAPEDLEGYERFHRDAQAIFERGFLELGYTHFGDVPTMLGAVPDLLRLDAVRTLFSFTSKYFRSDKMRQVFSFETLLVGGNPLSVPAIYAMIHFVEKTWGIHYAMGGTGALVQAMVRKFEELGGTVRYDAGVEEILVTDEWGQLVRTPLGRRQARGVRLESGETVPADLVVSNGDWANTYLRRVPRAARLVNSDLRVRAARQSMSLLVIYFGFRDNGRPLGLRHHNILLGPRYEALLREIFGQKVLGADFSQYLHVPTLTDPSLAPEGHHAAYTLVPVPHNGSGLDWTVQGPLLVDRVLNYLEERGVIPDLRARLTHLEYVTPDYFETTLDSYLGNAFGPEPVLTQSAFFRPHNRSEDVSNLYLVGAGAQPGAGTPSVMMSAKMTARLIAQDFGVPAEITG

SEQ ID NO: 16 - protein, 497 aa, Deinococcus gobiensis
MFDMGPTVITVPHFIEELFSLERDHAALNTPDYPPHTLSGERVKAGDSGGPRTREYVNLVPILPFYRIVFDDATFFDYDGDPVSTREQIARLAPEDLEGYERFHRDAQAIFERGFLELGYTHFGDLPTMLRVVPDLMKLDAVRTLFSFTSRYFSSDKMRQVFSFETLLIGGNPLSVPAIYAMIHFVEKTWGVHYAMGGTGALVQGFVRKFRELGGTVRYGTGVEEILVESGRGGPVRAPVGPRVARGVRLESGEELRADIVVSNGDWANTYLKRVPAAARLVNNDLRIKAAPQSMGLLVIYFGFRDDGQPLNLRHHNILLGPRYEALLREIFGKKVLGQDFSQYLHVPTLTDPALAPAGHHAAYTLVPVPHNGSGIDWSVEGPRLTERVLDYLEERGFIPDLRARLTHFEYVTPDYFEGTLDSYLGNAFGPEPVLAQSAFFRPHNRSEDVRGLYLVGAGAQPGAGTPSVMMSAKMTARLIAEDFGIHPDLLGAAADD

SEQ ID NO: 17 - protein, 555 aa, Deinococcus maricopensis
MRRKTAIIIGAGFGGLALGIRLQSLGFDTTIVEALDQPGGRAYQKRTPDGYVFDMGPTVITVPHFIEELFALERDGGRLHDPDFPEHVRHAERVREGDSGGPRTRDYVRLVPILPFYRIIFDDGTHFDYDGDPDSTRAQIRALAPEDLEAYERFHADARAIFERGFLELGYTHFGDLPSMLRVVPDLLRLDAVRTLFSFTRKYFRNPKMQQVFSFETLLVGGNPLSVPAIYAMIHFVEKTWGIHYAMGGTGALVDAFVRKYRELGGTLRLAAPVERILVTDDRGRPVRAPLGARVARGVRLQGGEELHADVVVSNGDWANTYMKLVEPRARLVNSDARVRLARQSMSLLVVYFGFRDDGRPLDLRHHNIILGPRYEGLLTDIFRNLHLADDFSQYLHVPTLTDPSLAPAGHHAAYTLVPVPHNGSGLNWAEVGPALVDRVLTFLDERGHIPNLRARLTHVSHITPDYFEGTLGAHLGNAFGPEPLLAQSAFMRPHNRSEDVRNLYLVGAGAQPGAGTPSVMMSAKMTARLIAQDFGVHADLVHGSAAVAVGAEPR

SEQ ID NO: 18 - protein, 573 aa, Deinococcus peraridilitoris
MTPQQQTPQAQSRSASRSGRSKTAVIIGAGFGGLALGIRLQSLGFDTTIMERRDAPGGRAYQFQADGFTFDMGPTVITVPHFIEELFSLERGKAGLLAPDFPPAVVSAPRVMSGLSGGPATRRYVDIVPILPFYRIYFDDGSFFDYDGDPEGTRAQIRVLAPEDLPGYERFHEDARAIFERGFLELGYTHFGDLGSMLRVVPDLLKLDAVRTLFSFARKYFRNPKMQQVFSFETLLVGGNPLKVPAIYAMIHFVEKTWGVHYVMGGTGALVRAFVRKFEELGGTMRYNAPVARIEVTDARGGMGGLFSRRFARGVTLQCGEHLAADVVVSNGDWANTYLKLIERRHRRVNSDARVKLARQSMSLLVVYFGFRADSLPLDLRHHNIILGPRYEELLTDIFDRKVLAEDFSQYLHLPTLTDPSLAPPGYHAAYTLVPVPHNGSGLDWTEVGPKLTERVLRFLEERGFIPGLRERLVYQHFVTPDYFEQELDSYQGNAFGVEPVLMQSAYFRPHNRSEDIGNLYLVGASAQPGAGTPSVMMSAKMTAREIARDFGVHSSILQSAFPQEDPEVPVRG

SEQ ID NO: 19 - protein, 562 aa, Deinococcus proteolyticus
MTSSHISSRTAPLRKTALIIGSGIGGLSLGIRLQSLGFDTRILERLDAPGGRAYQKRTPDGYVFDMGPTVLTVPHFIEELFALERDQAALDAADYPPSTLSGERVREGVSGGPRTSDYVQLVPILPFYRIYFDDGTYFDYDGDPEHTRQQIAELAPEDLEGYEEFNRDARAIFERGFLELGYTHFGDPQTMLAVVPDLLRLDAVRTLFGFTSKYFASDKMRQVFSFETLLVGGNPLSVPAIYAMIHFVEKTWGIHYAMGGTGALVRALVRKYEELGGQIRYGAGVEEILTMPRPGLLGRPAAQGVRLEGGEELHADIVVSNGDWANTYLKRLPAAARRVNSDLRVRAAAQSMGLVVVYFGFRDEGYAPLDLRHHNIILGPRYEELLGEIFSGGVLGEDFSQYLHIPTLTDPSLAPAGHHAAYTLIPVPNKSGRGAALNWENEGPELVKRVMEFLEERGYIPHLAERLTHLEWITPDYFDQTLDSFVGNAFGPEPKLIQSAYFRPHNRSEDVAGLYMVGASAQPGGGTPSVMMSAKMTARLIAEDFGIHPEIAAGVPAGYPTE

SEQ ID NO: 20 - protein, 578 aa, Deinococcus puniceus
MTHTPPSNPTQSNSTQRKTALIIGSGIGGLSLGIRLQSLGFNTTILEKLDAPGGRAYQKRTEDGYVFDMGPTVITVPHFIEELFALERDRAALTTPDYPAHVLENERVREGESGGPRTRDYVKLVPILPFYRIYFDDGTYFDYDGDPDSTRRQILELAPDDLAGYERFHKDAGAIFERGFLELGYTHFGDMPTMLRVVPDLMKLDAVRTLFSFTSKYFQSPKLRQVFSFETLLVGGNPLSVPAIYAMIHFVEKTWGIHYAMGGTGALVRAFVQKFEELGGQIRYGAEVERVLVSDAKGKPVKRPTGARVARGVALAGGEELHADITVSNGDWANLYLKLVPPQARLVNSDVRVRAARQSMSLLVIYFGFRRDGPNDRPLDLRHHNIILGPRYEELLTEIFGQKVLSKDFSQYLHVPTLTDPTLAPEGHHAAYTLVPVPHNASGLDWSVEGPKLVERVYDFLEERGYIPNLRERLTHSEYITPDYFEGTLDAYMGNAFGPEPTLIQSAYFRPHNRSEDVRNLYLVGAGAQPGAGTPSVMMSAKMTARLIAQDFGIHPDILGGPDEVAAQAEPDAVGAAD

SEQ ID NO: 21 - protein, 548 aa, Deinococcus radiodurans
MTSALPRPAPSPYARRKTALVIGAGFGGLALGIRLQSLGFDTTILERLDGPGGRAYQKRTPDGYVFDMGPTVLTVPHFIEELFALERDRAGLDAPDYPPEVLSGERVKEGVSGGPHTSRYVTLVPILPFYRIVFHDGTYFDYDGDPESTRRQIAELAPGDLAGYERFHADAEAIFRRGFLELGYTHFGDVPTMLRVVPDLLKLDAVRTLFSFTSKYFQSDKLRQVFSFETLLVGGNPLSVPAIYAMIHFVEKTWGIHYAMGGTGALVRGLVQKFEELGGAIRYGAGVDEVLVDGNLPGKRTARGVRLESGEELRADLVASNGDWANTYLKRVRPSARLVNSDLRVKAASESMSLLVVYFGFRGGDDLPLKHHNILLGPRYEALLSEIFGTKRLGEDFSQYLHVPTLTDPALAPAGHHAAYTLVPVPHNGSGIDWDVEGPKLAEAALADIERRGLIPGLRERLTHFEFITPDYFAGTLDSYLGNAFGPEPRLVQSAFFRPHNRSEDLHNFYLVGAGAQPGAGTPSVMMSAKMTARLIAEDFGIHADIRR

SEQ ID NO: 22 - protein, 567 aa, Deinococcus soli
MTPDLTATPAATRKTALIVGAGIGGLSLGIRLQSLGFDTTIVERLDQPGGRAYQKRTADGYVFDMGPTVITVPHFIEELFALERDKGMLGEADYPAQVLAPDARVREGESGGERTRDYVKLVPILPFYRIYFDDGTFFDYDGDPVSTRRQIADLAPEDLAGYERFHADAQAIFERGFLELGYTHFGDMPTMLRVVPDLMRLDAVRTLFSFTSKYFRNPKMRQVFSFETLLVGGNPLSVPAIYAMIHFVEKTWGIHYAMGGTGALVDAFVRKFEELGGTLRLNAGVQEILVTDDQGRPVRRPGGKRVARGLRLESGEELHADIVVSNGDWANTYLKRVPAAARLVNSDVRVKAARQSMSLLVIYFGFRREGPELNLRHHNIILGPRYEELLTEIFGKKVLGRDFSQYLHVPTLTDPTLAPEGHHAAYTLVPVPHNASGLDWNVEGPKLVERVYDFLEERGYIPNLRARLTHSEFITPDYFEGTLDSYLGNAFGPEPILAQSAYFRPHNRSEDVRNLYMVGAGAQPGGGTPSVMMSAKMTARLIAQDFGIHPSVRDGVPESRPAELAAD

SEQ ID NO: 23 - protein, 571 aa, Deinococcus swuensis
MTSPSLNTSRRKTALIIGSGIGGLSLGIRLQSLGFDTTILERLDAPGGRAYQKRTEDGYVFDMGPTVITVPHFIEELFALERDKGMLGEPDYPPHTLTEDARVKSGESGGPRTREYVKLVPILPFYRIYFDDGSFFDYDGDPESTRRQIGELAPEDLAGYEHFHADARAIFERGFLELGYTHFGDMTSMLRVVPDLMRLDAVRTLFSFTSKYFQNPKMRQVFSFETLLVGGNPLSVPAIYAMIHFVEKTWGIHYAMGGTGALVRAFVRKFEELGGTLRLNAGVDEILVTDGRGQPVKRPLGKRTARGVRLESGEELHADIVVSNGDWANTNLKRIPAAARLVNSDVRVKAARQSMSLLVIYFGFRDDPARPLQLRHHNIILGPRYEELLTEIFGQKVLGQDFSQYLHVPTLTDPSLAPAGHHAAYTLVPVPHNASGLDWAVEGPKLVDRVYSFLEERGYIPNLRERLTHSEYITPDYFAQTLDSYLGNAFGPEPVLIQSAYFRPHNRSEDIGNLYLVGAGAQPGGGTPSVMMSAKMTARLIADDFGIHKDVRDGVPAAAEALAREVAGATD

SEQ ID NO: 24 - DNA, 1902 bp, Deinococcus geothermalis
atgagtgagccaactgccaacctccagcctgccagccgcaccccgctgctggaccgggtgaatggcccggaagacctcaaacggctcggacgcgaccagctaccccagctcgctgccgaactgcgcgaggagatcgtgcgggtctgctcggtaggggggttacatctcgccagctctctgggcgcgaccgacctaatcgtggcgctacattacgtgctgcattcgccgcgcgaccgcattctgttcgacgtggggcatcaggcctacgcccacaagatgctcacgggccgccgccacctgatgcacaccgtcaagaaggagggcgggctgtcgggcttcaccaaggtgagcgagtccgaacacgacgccatcacggtgggccatgccagcacctccctcgccaatgcgctgggcatggcgcttgcacgggacgctttgggacaggattacaaggtggctgccgtgatcggggacggctcgctgacgggcggcatggcgctggcggcgctgaataccatcggggacctggggcggcgaatgctcattgtgcttaacgacaacgagatgagcatcagcgagaacgtgggggccatcaaccgcttcatgcggggtctccaggtgcagaagtggttccaggagggcgaggaagccgggaaaaaggccgtgcaggcggtcagcaagccgctcgccaacttgatgagccgcgccaagagttccacgcggcactttttcgatcccgccagtgtcaatccctttgccgcgatgggcgtgcgctatgtgggaccggtggacggccacaacgtgcaggaattggtgtggctgatcgagcggctggtcgacctcgatgggccgaccattctgcacgtcgtcaccaaaaagggcaagggcctgagctacgccgaggccgacccgatcaaatggcatggcccgggcaagtttgacccggcgacgggtgagtcggtgcccagcaatgcctactcgtggagcagcgcctttggagacgcggtgaccgagcttgcacggctggacccccgtacctttgtgatcacgcccgcgatgcgcgagggcagcggcctggtgcgctacagccaggttcacccccaccgttacctggatgtcggtatcgcggaggacgtggccgtcaccacggccgccggaatggcgcttcaggggatgcggcccatcgtggcgatctactccactttcctgcaacgcgcctacgatcaggtgctgcacgacgtcgccatcgagaatctgaacgtgaccttcgccatcgaccgtgggggcatcgtgggcgcagacggagccacccacaacggcgtcttcgacctgagttacctgcgctcgattccgaatgtcggcattggcctgccgaaggacgccgccgagctgcgcgggatgctgaagtatgcccaggagcatgctggccccttcgccatccgctatccgcgcggcaacgtggaacgcgtgccggaaggcacctggccggagctgaggtggggcacctgggaacgcttgcaagacggcgacgacgtggtgattctggcgggaggcaaggcgctggagtacgcgctgaaggccgcccgcgacctccccggcgtgggcgtggtgaatgcccgtttcgtgaagccgctcgaccaagggatgctgcgcgaggtggcgaccaaagcccgcgcactggtcacggtggaggacaacacggtcgtcggtgggttcggaagtgccgtcctggaagccctcagcgcgctggggctgagaaccccggtgcgggttctcggcatccccgacgcgtttcaggatcacgcgaccgtagagagcgtgcatgcccgtgcggggattgacgcgcctgccatccgcacggtcctggccgaacttggcgtggacgtgccgctggaggtctag

SEQ ID NO: 25 - DNA, 1893 bp, Deinococcus yunweiensis
atgacatccagcgacctcgtccccgttccgcagcgcctgctcgacgcggtgaactcgccggacgacctcaagaccctgaagcgcgagcagctgccgcaggtggcgcaggaactccgcgacgagatcgtgcgggtgtgctcggtgggtggcctgcacctggcgtcctcgctgggcgcgaccgacgtgatcgtggcgctgcactacgtcctgaactcgccgcgtgaccggattctgttcgatgtgggtcaccaggcctacgcgcacaagatgctcaccggccgccgcgagcagatggtcagcgtgaagaaggagggcgggctctcgggcttcaccaaggtcagcgagtcgccgcacgacgcgatcacggtggggcacgccagtaccagcctggcaaacgcgctgggcatggcgatggcacgggacgccctggggcaggattaccacgtggccgccgtgatcggtgacggcagcctgaccggcggcatggccctggccgccctaaacaccatcggggacacgcagcgcaagatgctgatcgtcctgaacgacaacgagatgagcatctccgagaacgtgggggccatgaacaagttcatgcgtggcctccaggtgcagaagtggttccaggagggcgagggcgcgggcaagaaggccatgcaggcggtcagccgcccgctcgccaacttcatgagccgcgccaagagcagcacccggcatttcttcgaccccgccagcgtgaatccctttgccaccatgggcgtgcgttatgtggggccagtcgacggacacaacgtgcaggaactcgtgtggctgctcgaacgcctcgtggaactcgacgggccgacgatcctgcatgtggtgaccaagaagggcaagggcctgagctacgccgaggccgatccgatctactggcacggccccggcaagttcgacccggagaccggggacttcgtgcccagcaatgcgtactcgtggagcaacgccttcggagacgccgtcacggagctggcgaaagccgatccccgcaccttcgtgatcacccccgccatgcgcgagggcagcgggctggtcggctacagcaaggcccatccgcaccgctatctggacgtcggcatcgccgaggaggtcgctgtgacggccgccgccggcatggccctccaggggctgcggcccgtcgtggcgatctactccaccttcctgcaacgcgcctacgaccaggtgctgcacgacgtcgccattgagcacttgaacgtcaccttcgctattgaccgggccgggatcgtcggcgcggacggggccacccacaacggcgtcttcgacctgagcttcctgcgctcgatcccaggtgtgcggatcggcctgcccaaggacgcgaccgagctgcgtggcatgctgaagtacgcccaggagcaccccggccccttcgccatccgctatccgcgtggcaccaccgagcgcgtgccagagggcacctggcccaccctggcgtggggcacgtgggagcgcgtgaagtccggcgacgacgtggtcatcctggcgggtggcaagggcctggagtatgcccagaaggccgccgccgacctgcccggcgtgggtgtcgtgaacgcccgtttcgtcaagccgcttgacgacgccatgctgcgagaggtggccggcagtgctcgcgctatcgtcaccgtcgaggacaacaccgtcgtcgggggatttggcagcgccgtgctggaggccctgaacgcctggggcctgaccgtgcccgtgcgcgtgctgggcatcccggacgaattccaggaacacgccaccgtggacagcgtgcatgcccgcgccggcatcgacgctcccgctatccgcacggtgctggccgagcttggggtggacgtgccgctgggcgtgtaa

SEQ ID NO: 26 - DNA, 1911 bp, Deinococcus radiopugnans
ttgaccgacatcaaaaaggctgatgggctgcacctcgggcacaagggcacgccgctgctggaccgcattgccggcccggctgacctcaagaagctctcgcgcgatcagttgcccgagctgagccaggaactgcgcgacgagatcgtgcgggtctgctcggtgggtgggctgcatctggcgtcctcgctgggggccaccgacctgatcgtggcgctgcattacgtgctgaacagtccgcgtgaccggattctcttcgacgtgggtcaccaggcctacgcccacaagatgctgaccgggcggcgggagcagatgcacaccgtcaagaaggaaggtgggctgagcggctttaccaaggtcagcgagtccgaacacgacgccattaccgtgggccacgccagcaccagcctggcgaacgcgctgggcatggcgatggcgagagacgcgctgggccaggactatcaggtggccgccgtgatcggcgacggctcgctgaccggcgggatggcgctggccgccctgaacaccatcggtgacctgcgccgcaagatgctgatcgtcctgaacgacaacgagatgagcatttccgagaacgtgggcgcgatcaacaagttcatgcgcggcctgcaggtccagaagtggtttcaggagggcgagggagccgggaaaaaggcggtgcagtccctgagcaagccgctggccgatttcatgagccgtgccaagagcagcacccgccacttcttcgatccggccagcgtcaatcccttcgccatgatgggcgtgcgttatgtcggcccggtcgacggccacaacgtccaggaactggtgtggctgatggaacggctggtggacctggacggccccacgatcctgcatgtcgtgacccgcaagggcaagggcctgagctacgccgaggccgacccgatctactggcacggtcccggtcaatttgacccggacaccggggatttcaaggccagcagcgcgtactcgtggagcgccgcgttcggcgacgccgtgaccgaactggccaaacgcgatccgcgtactttcgtgatcaccccggccatgcgcgagggcagcgggctggtgggctacagccgggcgcacccgcaccgttacctggacgtgggcatcgccgaggacgtggccgtgaccactgccgccggcatggcgttgcagggcctgcggcccatcgtggcgatctactccaccttcctgcaacgcgcctacgatcaggtgttgcacgacgtcgccatcgagaacctgaacgtcaccttcgccatcgaccgcgccgggatcgtgggggccgacggctcgacgcacaacggcgtgttcgacctgagctacctgcgcagcattcccaacgtgcggatcggcctgccgaaggacgcccacgagatgcgcggcatgctgaagtacgcgcaggagcacgacggtcccttcgccatccgttacccgcgcggcaacacggtcaaggtgccggaaggcacgtggcccacgctggaatggggcacgtgggagcgcgtcaaggaaggctccgacgccgtgatcctggccggcggcaaggcgctggaatacgcgcaggcggcggcggcggacctgcccggcgtcggtgtggtgaacgcccgtttcgtcaagccgctggacctgaacatgctgcgcgagctggccggcagcgcccgcacaatcatcaccgtggaggacaacacgctggtgggcggcttcggcagcgccgtgttggaggccctgaacagcatgggcttaaaggtgcccgtgcggacgctgggcattccggacgagttccaggaccacgccaccgccgagagcgtccacgcccgcgccggcatcgatgcccaggcgatccgcaccgtgctggccgagcttggggtggatgtgcctctgggcgtctga

SEQ ID NO: 27 - DNA, 1041 bp, Deinococcus geothermalis
gtgaccgaccccgacccgaacgctgccgcgcccacaggcctcccggcacgcaagctgcggcatcttgaagcctgtctgcgcccggaaagtcagtacatgggcgtgactaccgggctggagcgggtgccctggccttaccgggcgctcccagagctggacctggaggcggtggacctcacgacgaccttcctgggtcggagactgcgtgccccggtgttgattggagcgatgacgggaggcgcgcagcgggcggaggtcatcaatcgcaacctcgccaccgccgcggagcggttgggcatcgggatgatgttggggtcacagcgagtgatgctggagcgccctgaagcagcggtcagctttcgcgtgcgcgacgtggcccccggtgtactgctgctgggaaatctcggagcggcacagtttctgctggggtacggcgtgtccgaggccgagcgggcggtgcgggcggtggaggcagacggcctcgccatccatctcaacccgctgcaagaagccatgcaggccggtggggacacgcgctggcgaggtctcgcggcgcggctggccgaggtggtgcccgctctgccttttccggtgattctcaaggaggtggggcatggcctggatccggccactgtgcagaccgtggccacggcggggtttgcggcgctggacgtggcgggcgcgggcggtacgagttgggcgcgggttgagcaactggtgcgctacggcgccgttctcgcgccggacctgtgcgaagtgggactgcccaccgcaccggccatcgtggaggcgcgccgggcagctcccggaacacccctgatcgcctccgggggcatccgcaccggtttggacgcagcgcgtgccctggccctcggcgcgcaggtggtggcggtggctcggcctctcctcgctcccgcgctggagagtgcggcggcggtagaggcgtggcttgcgcggttcattcacgagctgcgggtggcgctgtttgtgggcggcttcaggagtgtggaggcggtgcggggccggctcgagctggtttga

SEQ ID NO: 28 - DNA, 1053 bp, Deinococcus yunweiensis
gtggacgactcgacgagcggcatccaggcacgcaagatgcaccatctccaagcgtgtctggagccgcacagccagtaccagacggtcacgaccggcctggacgcggtgccctggccgtaccgggcgctgcccgaggtcgatctgccggccgtacagctccagacaagcttcctgggtcgccggctggccgcccccgtgctgatcggtgccatgaccggcggggccgagcgggcacggatcatcaaccgcaatctggcgatggctgcgcagcgcctgggcatcggcatgatgctcggctcgcagcgcgtcatgctggagcgcccggaggtgaccgagaccttccgcgtgcgcgatgtggccccggacatcctgctcgtcgggaatctgggggcggcgcagttcggcctgggctacggcgcggccgaggcgctccgcgccgtacagcagatcgacgccgacgccctggcgattcatgccaatccccttcaggaagccatgcaggctgggggggacacgcgctggcggggcctgctggaacgcctgtcggacgtggtaccagcgctgccgttcccgacgatcctgaaggaagtcggacatgggctggatgtccacaccgcccacgcggcggccgacctgggcttcacggcgctggatgtggccggggccggtggcacgagctgggcgcgggtggagcagctcgtccgctacggcacggtgcgctcgccggccctgtgtgagctgggcatcccgaccgcgcgggccctgcgggacgtgcggggcgccctgcccggcatgccgctggtcgcgtccgggggcatccgcaccggcctggacgccgcccgtgccctggcgctgggggcgcaggtggtcgccattgcccggcccctgctggaaccggcattggacggcccggacgcggccgaggcgtggctcgcggccttcatccacgacctgcggatcgcgctgtttgtcggtggctacgacagtgtggacgaggtgcgcccggcgctggactggccgctggtgtcgggggctgtcagctctgggctgtga

SEQ ID NO: 29 - DNA, 504 bp, Deinococcus radiopugnans
atggtgcaggctctggaatggctcgatctggtagacgagaatgatcaggtggtcggccagatcacccgtgacgacgcctgggcacagcggcgggcagtgcggatggtcaacgcctttctggtcaaccggcgcggtgagctgtggattccgcgccgcaccgcctccaaacggatgtttcccaactgtctgaatatgagcgtgggaggccacgtcgaacgcggcgaaacctatctcagcgccttcaagcgcgagacgcacgaggaactgaatctgaacgtggacgcgctggactggcggaaaatcgccgccttttcgccgttcgagacgggactgagcagcttcatgcgcgtctatgagatcaggactgacgccgcgcacgacttcaattccgccgacttcagcgaggcgtggtggctgacgcccgccgaactgctggactggattgaggcgggcgaaccggccaagggtgatctggccgagctggtgcggcggtgctttccatga

SEQ ID NO: 30 - protein, 636 aa, Artificial Sequence
  R244C mutant of the DXP synthase from D. radiopugnans
MTDIKKADGLHLGHKGTPLLDRIAGPADLKKLSRDQLPELSQELRDEIVRVCSVGGLHLASSLGATDLIVALHYVLNSPRDRILFDVGHQAYAHKMLTGRREQMHTVKKEGGLSGFTKVSESEHDAITVGHASTSLANALGMAMARDALGQDYQVAAVIGDGSLTGGMALAALNTIGDLRRKMLIVLNDNEMSISENVGAINKFMRGLQVQKWFQEGEGAGKKAVQSLSKPLADFMSRAKSSTCHFFDPASVNPFAMMGVRYVGPVDGHNVQELVWLMERLVDLDGPTILHVVTRKGKGLSYAEADPIYWHGPGQFDPDTGDFKASSAYSWSAAFGDAVTELAKRDPRTFVITPAMREGSGLVGYSRAHPHRYLDVGIAEDVAVTTAAGMALQGLRPIVAIYSTFLQRAYDQVLHDVAIENLNVTFAIDRAGIVGADGSTHNGVFDLSYLRSIPNVRIGLPKDAHEMRGMLKYAQEHDGPFAIRYPRGNTVKVPEGTWPTLEWGTWERVKEGSDAVILAGGKALEYAQAAAADLPGVGVVNARFVKPLDLNMLRELAGSARTIITVEDNTLVGGFGSAVLEALNSMGLKVPVRTLGIPDEFQDHATAESVHARAGIDAQAIRTVLAELGVDVPLGV

SEQ ID NO: 31 - protein, 630 aa, Artificial Sequence
  R238C mutant of the DXP synthase from D. yunweiensis
MTSSDLVPVPQRLLDAVNSPDDLKTLKREQLPQVAQELRDEIVRVCSVGGLHLASSLGATDVIVALHYVLNSPRDRILFDVGHQAYAHKMLTGRREQMVSVKKEGGLSGFTKVSESPHDAITVGHASTSLANALGMAMARDALGQDYHVAAVIGDGSLTGGMALAALNTIGDTQRKMLIVLNDNEMSISENVGAMNKFMRGLQVQKWFQEGEGAGKKAMQAVSRPLANFMSRAKSSTCHFFDPASVNPFATMGVRYVGPVDGHNVQELVWLLERLVELDGPTILHVVTKKGKGLSYAEADPIYWHGPGKFDPETGDFVPSNAYSWSNAFGDAVTELAKADPRTFVITPAMREGSGLVGYSKAHPHRYLDVGIAEEVAVTAAAGMALQGLRPVVAIYSTFLQRAYDQVLHDVAIEHLNVTFAIDRAGIVGADGATHNGVFDLSFLRSIPGVRIGLPKDATELRGMLKYAQEHPGPFAIRYPRGTTERVPEGTWPTLAWGTWERVKSGDDVVILAGGKGLEYAQKAAADLPGVGVVNARFVKPLDDAMLREVAGSARAIVTVEDNTVVGGFGSAVLEALNAWGLTVPVRVLGIPDEFQEHATVDSVHARAGIDAPAIRTVLAELGVDVPLGV

SEQ ID NO: 32 - protein, 633 aa, Artificial Sequence
  R241C mutant of the DXP synthase from D. geothermalis
MSEPTANLQPASRTPLLDRVNGPEDLKRLGRDQLPQLAAELREEIVRVCSVGGLHLASSLGATDLIVALHYVLHSPRDRILFDVGHQAYAHKMLTGRRHLMHTVKKEGGLSGFTKVSESEHDAITVGHASTSLANALGMALARDALGQDYKVAAVIGDGSLTGGMALAALNTIGDLGRRMLIVLNDNEMSISENVGAINRFMRGLQVQKWFQEGEEAGKKAVQAVSKPLANLMSRAKSSTCHFFDPASVNPFAAMGVRYVGPVDGHNVQELVWLIERLVDLDGPTILHVVTKKGKGLSYAEADPIKWHGPGKFDPATGESVPSNAYSWSSAFGDAVTELARLDPRTFVITPAMREGSGLVRYSQVHPHRYLDVGIAEDVAVTTAAGMALQGMRPIVAIYSTFLQRAYDQVLHDVAIENLNVTFAIDRGGIVGADGATHNGVFDLSYLRSIPNVGIGLPKDAAELRGMLKYAQEHAGPFAIRYPRGNVERVPEGTWPELRWGTWERLQDGDDVVILAGGKALEYALKAARDLPGVGVVNARFVKPLDQGMLREVATKARALVTVEDNTVVGGFGSAVLEALSALGLRTPVRVLGIPDAFQDHATVESVHARAGIDAPAIRTVLAELGVDVPLEV

SEQ ID NO: 33 - protein, 329 aa, Deinococcus geothermalis
MHPDLLPRVLSLLPNRGDRPELRAFSAMLRDYPERGGKGIRSELLLASARAHGALPSTSAWEGALWLAAGLELFQNWVLIHDDIEDDSEERRGRPALHRLYGVPVAINVGDALHASMWAAVHRAGVPGGLEEFLNMVWRTAEGQHLDLTWVQERSWNLGEADYLAMVRLKTALYTVVVPLRLGALAAGVLPDERFTAAGEALGAAFQIRDDVLNLAGDPAKYGKEIGGDLWEGKRTLIVLHWLAHAPEEQRQVFLEQMRRDRADKDAAAIAAIHRWLLESGSVQYAQAYADTQAREGLARLTEALENAPDPQAARALLAQLRGLATREA

SEQ ID NO: 34 - protein, 329 aa, Deinococcus radiodurans
MRPELLARVLSLLPETSATPELARFYALLRDYPQRGGKGIRSELLLASARAHGLSESDTGWESALWLAAALELFQNWVLVHDDIEDDSEERRGRPALHHLCGMPVALNVGDALHAYMWAAVGKANVPGAFEEFLQMVYRTAEGQHLDLAWVEGREWGLRPADYLQMVGLKTAHYTVIVPLRLGALAAGMAPQDAFTPAGLALGTAFQIRDDVLNLAGDPVKYGKEIGGDLLEGKRTLIVLDWLTTAPDDRKAIFLDQMRRHRADKDPAVIDEIHRWLLESGSVEAAQDYAQAQAAEGLDLLEKALADAPDAQAAAALLASVRELATREK

SEQ ID NO: 35 - protein, 329 aa, Deinococcus deserti
MRPDLLSRVLSLLPDGGTRPELRQFSEMLRDYPQRGGKGIRSELLLASAQAHGVAHDTDAWDQAQWLAAGLELFQNWVLIHDDIQDDSEERRGQPALHRLYGVPLAINVGDALHAYMWAAVHRAGVPGAMEEFLTMIHRTAEGQHLDLSWVEHREWKLREADYLEMVQLKTAHYTVIVPLRLGALAAGVQPASAFTNAGLALGSAFQIRDDVLNLLGDPLKYGKEIGGDLLEGKRTLIVLRWLAQAPAEQREVFLDQMRRDRAAKDPAVIAQIHRWLLDSGCVTAAQDYAAARAEEGLSLLSSALSGAPGQAAVQQILGAMRALATRDA